SEQUENCE LISTING (11 sequences)

SEQ ID NO: 1 - protein, 23 aa, Homo sapiens
DIVMTQSPDSLAVSLGERATINC

SEQ ID NO: 2 - protein, 14 aa, Homo sapiens
WYQQKPGQPPLLIY

SEQ ID NO: 3 - protein, 32 aa, Homo sapiens
GVPDRPFGSGSGTDFTLTISSLQAEDVAVYYC

SEQ ID NO: 4 - protein, 11 aa, Homo sapiens
FGQGQTKLEIK

SEQ ID NO: 5 - protein, 30 aa, Homo sapiens
QVQLVQSGAEVKKPQASVKVSCKASQYTFT

SEQ ID NO: 6 - protein, 14 aa, Homo sapiens
WVRQAPGQRLEWMG

SEQ ID NO: 7 - protein, 32 aa, Homo sapiens
RVTITRDTSASTAYMELSSLRSEDTAVYYCAR

SEQ ID NO: 8 - protein, 11 aa, Homo sapiens
WGQGTLVTVSS

SEQ ID NO: 9 - protein, 613 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 10 - protein, 4 aa, Pseudomonas aeruginosa
KDEL

SEQ ID NO: 11 - protein, 4 aa, Pseudomonas aeruginosa
REDL

(19) United States
(12) Patent Application Publication
(10) Pub. No.: US 2011/0171216 A1
(43) Pub. Date: Jul. 14, 2011

(54) MONOCLONAL ANTIBODIES SPECIFIC FOR PANCREATIC NEOPLASIA CELLS

(76) Inventors: Phillip R. Streeter, Portland, OR (US); Markus Grompe, Portland, OR (US); Karin M. Hardiman, Portland, OR (US); Brett C. Sheppard, Portland, OR (US); Christopher L. Corless, Portland, OR (US); Terry K. Morgan, Portland, OR (US)

(21) Appl. No.: 13/062,942

(22) PCT Filed: Sep. 9, 2009
(86) PCT No.: PCT/US2009/056388

§ 371 Date: Mar. 8, 2011

Related U.S. Application Data
(60) Provisional application No. 61/095,563, filed on Sep. 9, 2008.

(51) Int. Cl. A61K 39/395; G01N 33/53; C12N 9/96; C12N 5/10; C12N 5/16; C12N 5/02; C12N 15/63; C07K 16/18; C07H 21/04

(52) U.S. Cl. 424/133.1; 424/141.1; 435/7.1; 435/188; 435/325; 435/326; 435/375; 435/378; 435/320.1; 530/387.3; 530/388.1; 530/391.3; 530/391.7; 536/23.53

(57) ABSTRACT

Isolated monoclonal antibodies are disclosed herein that specifically bind a cell surface antigen expressed on the human pancreatic cancer cells or precancerous pancreatic cancer cells. Humanized forms of these antibodies, functional fragments of these antibodies, and hybridomas producing these antibodies are also disclosed. The antibodies can be conjugated to an effector molecule, such as a detectable marker, a therapeutic agent, or a toxin. The antibodies can be used for in vitro or in vivo pancreatic cancer diagnosis or disease monitoring via detection of pancreatic cancer cells or the pancreatic cancer cell surface antigen. Methods of treating a pancreatic cancer by administration of the antibodies are also disclosed.

### PRIORITY CLAIM

This claims the benefit of U.S. Provisional Application No. 61/095,563, filed Sep. 9, 2008, which is incorporated herein by reference in its entirety.

### FIELD

This application relates to the fields of cancer, specifically to antibodies that specifically bind an antigen expressed on the surface of human pancreatic cancer cells and precancerous pancreatic cells.

### BACKGROUND

Pancreatic ductal adenocarcinoma is the most common type of pancreatic cancer and is one of the most lethal of human solid cancers. Although the incidence of pancreatic adenocarcinoma is relatively low, with roughly 37,000 new cases diagnosed per year in the United States, the five-year survival rate following diagnosis is only 1-5%. As a consequence of the high mortality rate for patients with pancreatic adenocarcinoma, this cancer is the fourth leading cause of cancer-related deaths for men and the fifth leading cause of cancer-related deaths for women in the United States. Despite continuing substantive efforts to alter the disease course in patients with pancreatic adenocarcinoma, conventional therapies including radiation and/or chemotherapy have had little impact on this aggressive disease. At present, tumor resection during early stage disease is the only potentially curative option for these patients.

One reason for the low survival rate for patients with pancreatic adenocarcinoma is the inability to diagnose this cancer during early stage disease. With currently available technology, most patients are diagnosed with metastatic or locally advanced disease, and only 15% of newly diagnosed patients present with operable cancer. A secondary reason for low survival of patients with this cancer is that systemic therapies do not substantially impact the disease course. The early diagnosis of pancreatic adenocarcinoma is complicated by the relatively non-descript symptoms associated with early disease. At presentation, jaundice and/or pain are frequently associated with this disease; and weight loss, abdominal mass, steatorrhea, and early satiety are also observed. Unfortunately, these symptoms are usually associated with advanced disease. The inability to detect this cancer at early stages is a significant barrier to the effective treatment of these patients, thus the need exists for reagents and assays for use in early detection of pancreatic adenocarcinoma.

The diagnosis of pancreatic adenocarcinoma is currently made using computed tomography (CT) scanning, endoscopic ultrasound (EUS), and fine needle biopsy (FNA) of the pancreatic lesion. The diagnosis is made based on the histologic appearance of cells in the fine needle biopsy and by results of the CT scan. However, imaging tests and EUS guided FNA have significant limitations. A mass may or may not be neoplastic, and EUS guided FNA is only about 80-90% sensitive for pancreatic cancer. Further, the negative predictive value of EUS with FNA is not 100%, as negative EUS with FNA may be either a true negative (where the patient does not have cancer) or a false negative (where the patient does have cancer). With currently available technology, the diagnosis of pancreatic adenocarcinoma is believed to be indeterminate approximately 30% of the time.

The treatment for early pancreatic cancer (including indeterminate cases) is a long surgery with a high rate of morbidity. Historical data reveal that 5-10% of patients undergoing the Whipple procedure for suspected pancreatic cancer have benign pancreatic conditions. Thus, there is significant need for highly sensitive and specific diagnostic tests that will allow accurate detection of early pancreatic cancer. Such tests would facilitate early intervention, while limiting the risk of pancreatic resection to those individuals who could truly benefit from the procedure.

### SUMMARY

The isolated monoclonal antibody HPC2 1-B3 and the hybridoma that produces the monoclonal antibody HPC2 1-B3 are disclosed herein. Chimeric forms of this antibody humanized forms of this antibody, and functional fragments of these antibodies, are also disclosed. The antibody, chimeric form, humanized form or functional fragment of these antibodies can be conjugated to an effector molecule, such as a detectable marker, a therapeutic agent, or a toxin. Kits that contain the monoclonal antibody HPC2 1-B3, a chimeric form, or a humanized form thereof or functional fragments thereof are also disclosed. Nucleic acid molecules encoding the monoclonal antibody HPC2 1-B3, a chimeric form or humanized form thereof or functional fragments thereof are also disclosed.

The HPC2 1-B3 antibody specifically binds a cell surface antigen expressed on pancreatic neoplasm cells, such as intraductal papillary mucinous neoplasm (IPMN) cells and pancreatic adenocarcinoma cells. Thus, the HPC2 1-B3 antibody, chimeric form or humanized form thereof or functional fragment thereof can be used to detect pancreatic neoplasm cells, such as IPMN cell and pancreatic adenocarcinoma cells. Thus cancer can be detected in situ. The antibody can also be used to detect metastatic cancers, wherein the cancer has metastasized to another tissue, such as the liver.

Methods are disclosed for detecting and/or isolating pancreatic neoplasia cells. Such methods include contacting a biological sample with the monoclonal antibody HPC2 1-B3, a chimeric form or a humanized form thereof or a functional fragment thereof under conditions wherein an immune complex will form and detecting the formation of the immune complex. The formation and detection of the immune complex detects the presence of the pancreatic neoplasm cell or the HPC2 13 antigen derived from the neoplasm cell.

Methods for inhibiting the growth of a pancreatic neoplasm cell are also disclosed. The disclosed methods include contacting a pancreatic neoplasm cell (or sample containing a pancreatic neoplasm cell) with an effective amount of the monoclonal antibody HPC2 1-B3, a chimeric form or a humanized form thereof or a functional fragment thereof, thereby inhibiting the growth of the pancreatic neoplasm cell. Methods of treating a pancreatic neoplasia are also disclosed herein. The methods includes administering a therapeutically effective amount of the monoclonal antibody HPC2 1-B3, chimeric form or humanized form thereof or functional fragment thereof to a subject with pancreatic neoplasia, such as pancreatic cancer, for example pancreatic adenocarcinoma.

The foregoing and other features and advantages will become more apparent from the following detailed description of several embodiments, which proceeds with reference to the accompanying figures.

### BRIEF DESCRIPTION OF THE FIGURES

FIGS. 1A-1D are digital images of immunofluorescence using HPC2 1-B3 as the primary antibody and an anti-mouse Cy3 conjugated antibody as the secondary antibody on acetone fixed frozen sections demonstrating that the HPC2 1-B3 monoclonal antibody reacts with pancreatic adenocarcinoma and not with normal pancreas or pancreatitis specimens. The Cy3 signal is shown. FIG. 1A is tissue from a normal pancreas showing no HPC2 1-B3 reactivity. FIG. 1B is tissue from a subject's pancreas diagnosed with pancreatitis showing no HPC2 1-B3 reactivity. FIG. 1C is tissue from a pancreatic adenocarcinoma sample showing a high degree of HPC2 1-B3 antibody staining. FIG. 1D is tissue from a second pancreatic adenocarcinoma sample showing a high degree of HPC2 1-B3 antibody staining.

FIG. 2 is a digital image of an imunohistochemical stain of an intraductal papillary mucinous neoplasm (IPMN) using the HPC2 1-B3 antibody. Tissue sections were exposed to HPC2 1-B3 as the primary antibody and a peroxidase-conjugated second antibody. Peroxidase was detected using the substrate 3,3′-Diaminobenzidine which yields a brown precipitate. Sections were counterstained with hematoxolin. The HPC2 1-B3 antibody was detected on this section using a peroxidase-conjugated second antibody, and illustrates presence of the antigen on the duct luminal surface.

FIG. 3 is a set of dot plots from exemplary flow cytometry trials showing that the antigen recognized by the HPC2 1-B3 antibody is a cell surface antigen. Flow cytometry using HPC2 1-B3 as the primary antibody and an allophycocyanin (APC)-conjugated secondary antibody reveals that pancreatic adenocarcinoma cells and a pancreatic cancer cell line (Panc 1) express the HPC2 1-B3 antigen on their cell surface. Cells were also stained with propidium iodide, to exclude dead cells. The negative control treatment was cells exposed to an isotype matched primary antibody followed by the APC-conjugated secondary antibody. Pancreatic adenocarcinoma and Panc 1 cells were found to express the antigen recognized by the HPC2 1-B3 antibody, as evidenced by an upward shift in the staining profile of cells treated with HPC2 1-B3.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NOs: 1-8 are exemplary amino acid sequence of human MAb framework regions.

SEQ ID NO: 9 is an exemplary Pseudomonas exotoxin A (PE) amino acid sequence.

SEQ ID NOs: 10-11 are exemplary C-terminal PE amino acid sequences.

### DETAILED DESCRIPTION

### I. Abbreviations

APC: allophycocyanin
CDR: complementarity determining region
CT: computed tomography
DAPI: 4′,6-diamidino-2-phenylindole
dsFv: disulfide stabilized fragment of a variable region
ELISA: enzyme-linked immunosorbent assay
EM: effector molecule
Fab′: antigen binding immunoglobulin fragment
F(ab)′₂: divalent antigen binding immunoglobulin fragment
FACS: fluorescence activated cell sorting
FNA: fine needle biopsy
Fv: fragment of a variable region
GFP: green fluorescent protein
kDa: kilodaltons
LCDR: light chain complementarity determining region
HCDR: heavy chain complementarity determining region
IPMN: intraductal papillary mucinous neoplasia
Ig: immunoglobulin
MAb: monoclonal antibody
MRI: magnetic resonance imaging
PE: Pseudomonas exotoxin A
PET: positron emission tomography
scFv: single chain fragment of a variable region
SDR: specificity determining residues
SDS-PAGE: sodium dodecyl(lauryl)sulfate-polyacrylamide gel electrophoresis
EUS: endoscopic ultrasound
V_{H}: variable region of a heavy chain
V_{L}: variable region of a light chain
YFP: Yellow fluorescent protein

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” In case of conflict, the present specification, including explanations of terms, will control.

To facilitate review of the various embodiments of this disclosure, the following explanations of specific terms are provided:

Administration: The introduction of a composition into a subject by a chosen route. For example, if the chosen route is intravenous, the composition is administered by introducing the composition into a vein of the subject.

Amplification: Refers to use of a technique that increases the number of copies of a nucleic acid molecule in a sample, for example the amplification of a nucleic acid that encodes the monoclonal antibody HPC2 1-B3 a humanized form thereof or fragment thereof. An example of amplification is the polymerase chain reaction, in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification may be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in PCT Publication No. WO 90/01069; ligase chain reaction amplification, as disclosed in European patent publication No. EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Animal: Living multi-cellular vertebrate organisms, a category that includes, for example, mammals and birds. The term mammal includes both human and non-human mammals. Similarly, the term “subject” includes both human and veterinary subjects.

Antibody: A polypeptide ligand comprising at least a light chain or heavy chain immunoglobulin variable region which specifically recognizes and binds an epitope of an antigen or a fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

This includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds an antigen of interest has a specific V_{H} region and the V_{L} region sequence, and thus specific CDR sequences. Antibodies with different specificities (due to different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected, or a progeny thereof. Monoclonal antibodies are produced by methods known to those of skill in the art, for instance by making hybrid antibody-forming cells from a fusion of myeloma cells with immune spleen cells. Monoclonal antibodies include humanized monoclonal antibodies. In some example a monoclonal antibody is the monoclonal antibody HPC2 1-B3.

A “chimeric antibody” has framework residues from one species, such as human, and CDRs or SDRs (which generally confer antigen binding) from another species, such as a murine antibody that specifically binds a cell surface antigen on a pancreatic neoplasm cell.

A “humanized” antibody immunoglobulin is an immunoglobulin including a human framework region and one or more CDRs from a non-human or SDRs (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, such as at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs or SDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

Binding affinity: Affinity of an antibody, such as the monoclonal antibody HPC2 1-B3, for an antigen. In one embodiment, affinity is calculated by a modification of the Scatchard method described by Frankel et al., Mol. Immunol., 16:101-106, 1979. In another embodiment, binding affinity is measured by an antigen/antibody dissociation rate. In yet another embodiment, a high binding affinity is measured by a competition radioimmunoassay. In several examples, a high binding affinity is at least about 1×10⁻⁸ M. In other embodiments, a high binding affinity is at least about 1.5×10⁻⁸ M, at least about 2.0×10⁻⁸ M, at least about 2.5×10⁻⁸ M, at least about 3.0×10⁻⁸ M, at least about 3.5×10⁻⁸ M, at least about 4.0×10⁻⁸ M, at least about 4.5×10⁻⁸ M, or at least about 5.0×10⁻⁸ M.

Chimeric antibody: An antibody which includes sequences derived from two different antibodies, which typically are of different species. Most typically, chimeric antibodies include human and murine antibody domains, generally human constant regions and/or framework regions and murine variable regions, murine CDRs and/or murine SDRs. However, a chimeric antibody can also include chimpanzee antibody domains, such as chimpanzee constant regions and/or chimpanzee framework regions and murine variable regions. In some examples a chimeric antibody includes the SDRs or CDRs from the monoclonal antibody HPC2 1-B3.

Chemotherapeutic agents: Any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating pancreatic adenocarcinoma or another tumor. In one embodiment, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., ©2000 Churchill Livingstone, Inc; Baltzer, L., Berkery, R. (eds): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer. One example is the administration of an antibody or a fragment thereof that binds pancreatic adenocarcinoma used in combination with a radioactive or chemical compound.

Complementarity Determining Region (CDR): Amino acid sequences which together define the binding affinity and specificity of the natural Fv region of a native Ig binding site. The light and heavy chains of an immunoglobulin each have three CDRs, designated L-CDR1, L-CDR2, L-CDR3 and H-CDR1, H-CDR2, H-CDR3, respectively. By definition, the CDRs of the light chain are bounded by the residues at positions 24 and 34 (L-CDR1), 50 and 56 (L-CDR2), 89 and 97 (L-CDR3); the CDRs of the heavy chain are bounded by the residues at positions 31 and 35b (H-CDR1), 50 and 65 (H-CDR2), 95 and 102 (H-CDR3), using the numbering convention delineated by Kabat et al., (1991) Sequences of Proteins of Immunological Interest, 5^{th} Edition, U.S. Department of Health and Human Services, Public Health Service, National Institutes of Health, Bethesda, Md. (NIH Publication No. 91-3242). CDRs contain the specificity determining regions (SDRs) of the antibody. In some examples a CDR is a CDR from the monoclonal antibody HPC2 1-B3.

Contacting: Placement in direct physical association. Includes both in solid and liquid form.

Cytotoxicity: The toxicity of a molecule, such as an immunotoxin, to the cells intended to be targeted, as opposed to the cells of the rest of an organism. In one embodiment, in contrast, the term “toxicity” refers to toxicity of an immunotoxin to cells other than those that are the cells intended to be targeted by the targeting moiety of the immunotoxin, and the term “animal toxicity” refers to toxicity of the immunotoxin to an animal by toxicity of the immunotoxin to cells other than those intended to be targeted by the immunotoxin.

Effective amount or Therapeutically effective amount: The amount of agent, such as the antibodies or antibody fragments disclosed herein, that is an amount sufficient to prevent, treat (including prophylaxis), reduce and/or ameliorate the symptoms and/or underlying causes of any of a disorder or disease. In one embodiment, an “effective amount” is sufficient to reduce or eliminate a symptom of a disease, such as a pancreatic cancer.

Effector molecule: The portion of a chimeric molecule, for example a chimeric molecule that includes a disclosed antibody or fragment thereof, that is intended to have a desired effect on a cell to which the chimeric molecule is targeted. Effector molecules are also known as an effector moieties (EM), therapeutic agents, or diagnostic agents, or similar terms.

Therapeutic agents include such compounds as nucleic acids, toxins, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the molecule linked to a targeting moiety, such as an antibody, may be an encapsulation system, such as a liposome or micelle that contains a therapeutic composition such as a drug, a nucleic acid (such as an antisense nucleic acid), or another therapeutic moiety that can be shielded from direct exposure to the circulatory system. Means of preparing liposomes attached to antibodies are well known to those of skill in the art. See, for example, U.S. Pat. No. 4,957,735; and Connor et al., Pharm. Ther. 28:341-365, 1985. Diagnostic agents or moieties include radioisotopes and other detectable labels. Detectable labels useful for such purposes are also well known in the art, and include radioactive isotopes such as ³²P, ¹²⁵I, and ¹³¹I, fluorophores, chemiluminescent agents, magnetic resonance imaging agents and enzymes.

Epitope: An antigenic determinant, for example an antigenic determinant present on the surface of a pancreatic cancer cell or precancerous pancreatic cell. These are particular chemical groups or peptide sequences on a molecule that are antigenic, for example elicit a specific immune response. An antibody specifically binds a particular antigenic epitope.

Expressed: Translation of a nucleic acid into a protein. Proteins may be expressed and remain intracellular, become a component of the cell surface membrane, or be secreted into the extracellular matrix or medium. In some examples, a disclosed antibody or fragment thereof is expressed from a nucleic acid sequence, for example expressed from an expression vector.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence, for example the transcription and translation of the nucleic acid sequence encoding a disclosed antibody or fragment thereof from an expression vector, for example from a host cell transformed with an expression vector. Expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, pap, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Framework Region: Amino acid sequences interposed between CDRs, and includes variable light and variable heavy framework regions. The framework regions serve to hold the CDRs of an antibody or fragment thereof in an appropriate orientation for antigen binding.

Heterologous: A heterologous sequence is a sequence that is not normally (in the wild-type sequence) found adjacent to a second sequence. In one embodiment, the sequence is from a different genetic source, such as a virus or organism, than the second sequence. For example a nucleotide sequence encoding a HPC2 1-B3 antibody can be connected to a heterologous nucleotide sequence encoding toxin.

Host cells: Cells in which a vector can be propagated and its DNA expressed, for example a vector encoding a disclosed antibody of fragment thereof. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. In some examples, a host cell propagates a vector encoding a HPC2 1-B3 antibody, functional fragment thereof, or a humanized form thereof.

Immunoconjugate: A covalent linkage of an effector molecule to an antibody, such as a HPC2 1-B3 antibody. The effector molecule can be a detectable label or an immunotoxin. Specific, non-limiting examples of toxins include, but are not limited to, abrin, ricin, Pseudomonas exotoxin (PE, such as PE35, PE37, PE38, and PE40), diphtheria toxin (DT), botulinum toxin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain Ia of PE and the B chain of DT) and replacing it with a different targeting moiety, such as an antibody. A “chimeric molecule” is a targeting moiety, such as a ligand or an antibody, conjugated (coupled) to an effector molecule. The term “conjugated” or “linked” refers to making two polypeptides into one contiguous polypeptide molecule. In one embodiment, an antibody is joined to an effector molecule (EM). In another embodiment, an antibody joined to an effector molecule is further joined to a lipid or other molecule to a protein or peptide to increase its half-life in the body. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because immunoconjugates were originally prepared from two molecules with separate functionalities, such as an antibody and an effector molecule, they are also sometimes referred to as “chimeric molecules.” The term “chimeric molecule,” as used herein, therefore refers to a targeting moiety, such as a ligand or an antibody, conjugated (coupled) to an effector molecule.

Immunologically reactive conditions: Includes reference to conditions which allow an antibody raised against a particular epitope to bind to that epitope (or cell expressing the epitope) to a detectably greater degree than, and/or to the substantial exclusion of, binding to substantially all other epitopes (or cells not expressing the epitope) Immunologically reactive conditions are dependent upon the format of the antibody binding reaction and typically are those utilized in immunoassay protocols or those conditions encountered in vivo. See Harlow & Lane, supra, for a description of immunoassay formats and conditions. The immunologically reactive conditions employed in the methods are “physiological conditions” which include reference to conditions (such as temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

Intraductal papillary mucinous neoplasm (IPMN): Intraductal tumors with variable amounts of papilla formation, mucin production, and cytoarchitectural atypia. IPMN can be classified into three classifications (or grades) of Pancreatic Intraepithelial Neoplasia (PanIN), PanIN-1, PanIN-2, and PanIN-3. PanIN-1A (Pancreatic Intraepithelial Neoplasia 1-A) are flat epithelial lesions composed of tall columnar cells with basally located nuclei and abundant supranuclear mucin. The nuclei are small and round to oval in shape. When oval the nuclei are oriented perpendicular to the basement membrane. It is recognized that there is considerable histologic overlap between non-neoplastic flat hyperplastic lesions and flat neoplastic lesions without atypia. PanIN-1B: (Pancreatic Intraepithelial Neoplasia 1-B) have a papillary, micropapillary or basally pseudostratified architecture, but are otherwise identical to PanIN-1A. PanIN-2 (Pancreatic Intraepithelial Neoplasia 2) have an architecture that may be flat or papillary. Cytologically, by definition, these lesions have some nuclear abnormalities. These abnormalities may include some loss of polarity, nuclear crowding, enlarged nuclei, pseudo-stratification and hyperchromatism. These nuclear abnormalities fall short of those seen in PanIN-3. Mitoses are rare, but when present are non-luminal (not apical) and not atypical. True cribriforming luminal necrosis and marked cytologic abnormalities are generally not seen, and when present should suggest the diagnosis of PanIN-3. PanIN-3 (Pancreatic Intraepithelial Neoplasia 3) Architecturally, these lesions are usually papillary or micropapillary, however, they may rarely be flat. True cribriforming, budding off of small clusters of epithelial cells into the lumen and luminal necroses should all suggest the diagnosis of PanIN-3. Cytologically, these lesions are characterized by a loss of nuclear polarity, dystrophic goblet cells (goblet cells with nuclei oriented towards the lumen and mucinous cytoplasm oriented toward the basement membrane), mitoses which may occasionally be abnormal, nuclear irregularities and prominent (macro) nucleoli.

Isolated: An “isolated” biological component (such as a nucleic acid, peptide, for example and antibody or fragment thereof (for example a HPC2 1-B3 antibody), cell or protein) has been substantially separated, produced apart from, or purified away from other biological components in the cell of the organism in which the component naturally occurs. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids. An isolated cell type (such as a pancreatic neoplasia cell) has been substantially separated from other cell types, such as a different cell type that occurs in an organ. A purified nucleic acid, peptide, cell or component can be at least 90%, at least 95%, at least 96%, at least 97%, at least 98%, or at least 99% pure.

Label: A detectable compound or composition that is conjugated directly or indirectly to another molecule, such as an antibody (for example a HPC2 1-B3 antibody) or a protein, to facilitate detection of that molecule. Specific, non-limiting examples of labels include fluorescent tags, enzymatic linkages, and radioactive isotopes.

Linker peptide: A peptide within an antibody binding fragment (such as an Fv fragment, for example a HPC2 1-B3 antibody fragment) which serves to indirectly bond the variable heavy chain to the variable light chain. “Linker” can also refer to a peptide serving to link a targeting moiety, such as a scFv, to an effector molecule, such as a cytotoxin or a detectable label.

The terms “conjugating,” “joining,” “bonding” or “linking” refer to making two polypeptides into one contiguous polypeptide molecule, or to covalently attaching a radionuclide or other molecule to a polypeptide, such as an scFv. In the specific context, the terms include reference to joining a ligand, such as an antibody moiety, to an effector molecule (“EM”). The linkage can be either by chemical or recombinant means. “Chemical means” refers to a reaction between the antibody moiety and the effector molecule such that there is a covalent bond formed between the two molecules to form one molecule.

Neoplasia: The process of abnormal and uncontrolled growth of cells. Neoplasia is one example of a proliferative disorder. The product of neoplasia can be a neoplasm (a tumor), which is an abnormal growth of tissue that results from excessive cell division. A tumor that does not metastasize is referred to as “benign.” A tumor that invades the surrounding tissue and/or can metastasize is referred to as “malignant.” In some examples, a neoplasm is a neoplasm of the pancreas. In a specific example a neoplasm of the pancreas is an intraductal papillary mucinous neoplasm (IPMN). In other examples, a neoplasm of the pancreas is pancreatic adenocarcinoma, such as a pancreatic ductal adenocarcinoma.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, for example an antibody, such as a HPC2 1-B3 antibody or a portion of an antibody, such as V_{H} or V_{L} HPC2 1-B3 antibody. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see for example, Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, such as version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information, for example on the world wide web at ncbi.nlm nih gov. The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Pancreatic cancer: A malignant tumor within the pancreas. The prognosis is generally poor. About 95% of pancreatic cancers are adenocarcinomas. The remaining 5% are tumors of the exocrine pancreas (for example, serous cystadenomas), ascinar cell cancers, and pancreatic neuroendocrine tumors (such as insulinomas). A pancreatic adenocarcinoma occurs in the glandular tissue. Symptoms include abdominal pain, loss of appetite, weight loss, jaundice and painless extension of the gallbladder.

Classical treatment for pancreatic cancer, including adenocarcinomas and insulinomas includes surgical resection (such as the Whipple procedure) and chemotherapy with agent such as fluorouracil, gemcitabine, and erlotinib.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. An “epitope” is a region of a polypeptide capable of binding an immunoglobulin generated in response to contact with an antigen.

Pharmaceutically acceptable carriers: The pharmaceutically acceptable carriers useful in conjunction with the antibodies and fragments thereof disclosed herein are conventional. Remington's Pharmaceutical Sciences, by E. W. Martin, Mack Publishing Co., Easton, Pa., 15th Edition (1975), describes compositions and formulations suitable for pharmaceutical delivery of the antibodies herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound, such as antibody, or fragment thereof disclosed herein, or a composition capable of inducing a desired therapeutic or prophylactic effect when properly administered to a subject or a cell.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, such as by genetic engineering techniques. Similarly, a recombinant protein is one encoded for by a recombinant nucleic acid molecule. A recombinant toxin is a chimeric protein in which a cell targeting moiety is fused to a toxin (Pastan et al., Science, 254:1173-1177, 1991). If the cell targeting moiety is the Fv portion of an antibody, the molecule is termed a recombinant immunotoxin (Chaudhary et al., Nature, 339:394-397, 1989). The toxin moiety is genetically altered so that it cannot bind to the toxin receptor present on most normal cells. Recombinant immunotoxins selectively kill cells which are recognized by the antigen binding domain, for example a recombinant immunotoxin that includes a HPC2 1-B3 antibody can be used to selectively kill cells that the HPC2 1-B3 antibody binds, for example intraductal papillary mucinous neoplasia (IPMN) cells and pancreatic adenocarcinoma cells.

Specific binding agent: An agent that binds substantially only to a defined target. Thus a pancreatic cancer cell specific binding agent is an agent that binds substantially to a pancreatic cancer cell and not to other cell types. In several embodiments, a specific binding agent is a monoclonal antibody, such as HPC2 1-B3 or a humanized form thereof or a functional fragment thereof.

The term “specifically binds” refers, with respect to a cell, such as a pancreatic endocrine cell, to the preferential association of an antibody or other ligand, in whole or part, with a cell or tissue bearing that antigen and not to cells or tissues lacking that antigen. It is, of course, recognized that a certain degree of non-specific interaction may occur between a molecule and a non-target cell or tissue. Nevertheless, specific binding may be distinguished as mediated through specific recognition of the antigen. Although selectively reactive antibodies bind antigen, they may do so with low affinity. On the other hand, specific binding results in a much stronger association between the antibody (or other ligand) and cells bearing the antigen than between the bound antibody (or other ligand) and cells lacking the antigen. Specific binding typically results in greater than 2-fold, such as greater than 5-fold, greater than 10-fold, or greater than 100-fold increase in amount of bound antibody or other ligand (per unit time) to a cell or tissue expressing the target epitope as compared to a cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Therapeutic agent: Used in a generic sense, it includes treating agents, prophylactic agents, and replacement agents. A therapeutic agent can be an antibody that specifically binds pancreatic cancer cells, such as pancreatic adenocarcinoma cells.

Toxin: A molecule that is cytotoxic for a cell. Toxins include abrin, ricin, Pseudomonas exotoxin (PE), diphtheria toxin (DT), botulinum toxin, saporin, restrictocin or gelonin, or modified toxins thereof. For example, PE and DT are highly toxic compounds that typically bring about death through liver toxicity. PE and DT, however, can be modified into a form for use as an immunotoxin by removing the native targeting component of the toxin (such as domain Ia of PE or the B chain of DT) and replacing it with a different targeting moiety, such as an antibody, for example a HPC2 1-B3 antibody.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (for example DNA-protein complexes, viral envelope/capsid-DNA complexes) and by biological infection by viruses such as recombinant viruses (see for example Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### III. Overview of Several Embodiments

Most patients diagnosed with pancreatic adenocarcinoma have a life expectancy of months rather than years. The poor prognosis for these patients is due to 1) the inability to detect early stage disease, 2) the metastasis of these tumors to distant sites early during the disease course, and 3) the resistance of the disease to conventional chemotherapy and/or radiation therapy. For patients with tumors located in the head and body of the pancreas, symptoms of disease are associated with compression of the bile duct, the pancreatic duct, the mesenteric and celiac nerves, and the duodenum; and these tumors may or may not cause the patient pain. For tumors located in the tail of the pancreas, patients may have pain on the left side of the abdomen, but pain is generally associated with late stage disease. Thus, patients with pancreatic adenocarcinoma do not generally seek treatment during early stage disease.

As patient survival depends on early detection of this disease, there is a need for diagnostic test for disease detection and diagnosis, for example early in disease such as at the stage of precancerous pancreatic lesions, such as intraductal papillary mucinous neoplasia (IPMN). Current serum markers of disease include: the sialylated Lewis^{a} blood group antigen CA19-9, macrophage inhibitory cytokine-1 (MIC-1; also known as placental TGF-beta, prostate-derived factor, and growth/differentiation factor 15); and osteopontin. Of these markers, CA19-9 has been the most widely studied. Unfortunately, the results from those studies do not support the use of this marker in disease detection and diagnosis, particularly in the diagnosis of early disease, where a high frequency of patients test negatively for CA19-9. In addition to the false-negative results associated with detection of early disease, false-positive results are obtained at high frequency in patients with acute cholangitis and chronic pancreatitis. Current data suggest that MIC-1 may be a better marker of pancreatic adenocarcinoma than CA19-9. However, MIC-1 is also present at high frequency in patients with pancreatitis, which could lead to misdiagnosis of pancreatitis as pancreatic cancer. Thus, prior to this disclosure there is no reported marker that can be used to accurately detect and diagnose early stage pancreatic adenocarcinoma and precancerous lesions of the pancreas.

A. Monoclonal Antibodies that Bind Pancreatic Neoplasia

Disclosed herein are isolated monoclonal antibodies (the HPC2 1-B3 antibody) produced by the hybridoma HPC2 1-B3, as Accession No. PTA-9400 deposited with the ATCC on Jul. 3, 2008, in accordance with the Budapest Treaty, humanized forms of the HPC2 1-B3 antibody, and functional fragments thereof. Methods of using these antibodies are also disclosed. The HPC2 1-B3 antibody specifically binds cell surface antigens present on pancreatic neoplasm cells, such as intraductal papillary mucinous neoplasm (IPMN) cells or pancreatic adenocarcinoma cells, for example ductal adenocarcinoma cells. As disclosed herein, the monoclonal antibody, HPC2 1-B3 selectively reacts with pancreatic adenocarcinoma cells as well as precancerous IPMN, but does not react with pancreatic cells in specimens of normal pancreas or pancreatitis. This antibody can also be used to detected metastatic cancers, such as metastatic pancreatic adenocarcinomas.

Generally, the monoclonal antibodies produced by the HPC2 1-B3 hybridoma include a variable heavy (V_{H}) and a variable light (V_{L}) chain and specifically bind the cell surface antigen. For example, the HPC2 1-B3 antibody can specifically bind pancreatic adenocarcinoma cells, and can bind the cell surface antigen of such cells with an affinity constant of at least 10⁶ M⁻¹, such as at least 10⁷ M⁻¹, at least 10⁸ M⁻¹, at least 5×10⁸ M⁻¹, or at least 10⁹ M⁻¹. The HPC2 1-B3 antibody can also specifically bind precancerous pancreatic cells, such as IPMN cells (for example precancerous lesions classified as PanIN 1-3), and can bind the cell surface antigen of such cells with an affinity constant of at least 10⁶ M⁻¹, such as at least 10⁷ M⁻¹, at least 10⁸ M⁻¹, at least 5×10⁸ M⁻¹, or at least 10⁹ M⁻¹. Hybridoma cells and their progeny that secrete the monoclonal antibody HPC2 1-B3 (deposited with the ATCC on Jul. 3, 2008, in accordance with the Budapest Treaty as Accession No. PTA-9400) are also encompassed by this disclosure.

The production of chimeric antibodies, which include a framework region from one antibody and the CDRs or SDRs from a different antibody, is well known in the art. Thus chimeric and humanized forms of the HPC2 1-B3 antibody are provided herein. These antibodies include the CDRs (or SDRs) of the HPC2 1-B3 antibody and framework regions from a different antibody. In one example, the framework regions are human. In some embodiments, a humanized antibody that specifically binds pancreatic neoplasia cells (for example IPMN cells and pancreatic adenocarcinoma cells, such as pancreatic ductal adenocarcinoma cells) is a humanized form of the HPC2 1-B3 monoclonal antibody or a functional fragment thereof. In one example the sequence of the specificity determining regions (SDRs) of each CDR from the HPC2 1-B3 monoclonal antibody is determined. Residues outside the SDRs (non-ligand contacting sites) can be substituted and the monoclonal antibody retains its ability to bind pancreatic neoplasia cells. Thus, the humanized or chimeric antibody can include all three heavy chain SDRs (or CDRs) and all three light chain SDRs (or CDRs) of the HPC2 1-B3 antibody. Functional fragments of these antibodies, that specifically bind a surface antigen present on pancreatic neoplasm cells, are also encompassed by this disclosure.

The antibody or antibody fragment can be a humanized immunoglobulin having complementarity determining regions (CDRs) from the HPC2 1-B3 monoclonal antibody and immunoglobulin and heavy and light chain variable region frameworks from human acceptor immunoglobulin heavy and light chain frameworks. Generally, the humanized immunoglobulin specifically binds to pancreatic adenocarcinoma cells or specifically binds to IPMN cells with an affinity constant of at least 10⁷ M⁻¹, such as at least 10⁸ M⁻¹ at least 5×10⁸ M⁻¹ or at least 10⁹ M⁻¹.

Humanized monoclonal antibodies can be produced by transferring donor CDRs from heavy and light variable chains of the donor mouse immunoglobulin (such as the HPC2 1-B3 monoclonal antibody) into a human variable domain, and then substituting human residues in the framework regions when required to retain affinity. The use of antibody components derived from humanized monoclonal antibodies obviates potential problems associated with the immunogenicity of the constant regions of the donor antibody. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. U.S.A. 89:4285, 1992; Sandhu, Crit. Rev. Biotech. 12:437, 1992; and Singer et al., J. Immunol. 150:2844, 1993. The antibody may be of any isotype, but in several embodiments the antibody is an IgG, including but not limited to, IgG₁, IgG₂, IgG₃ and IgG₄.

In one embodiment, the sequence of the humanized immunoglobulin heavy chain variable region framework can be at least about 65% identical to the sequence of the donor immunoglobulin heavy chain variable region framework. Thus, the sequence of the humanized immunoglobulin heavy chain variable region framework can be at least about 75%, at least about 85%, at least about 95%, or at least about 99% identical to the sequence of the donor immunoglobulin heavy chain variable region framework. The sequences of the heavy and light chain frameworks are known in the art. Human framework regions, and mutations that can be made in a humanized antibody framework regions, are known in the art (see, for example, in U.S. Pat. No. 5,585,089).

Exemplary human antibodies LEN and 21/28 CL are of use in providing framework regions. Exemplary light chain frameworks of human MAb LEN have the following sequences:

| | |
|---|---|
| FR1: | |
| DIVMTQS PDSLAVSLGERATINC | (SEQ ID NO: 1) |
| FR2: | |
| WYQQKPGQPPLLIY | (SEQ ID NO: 2) |
| FR3: | |
| GVPDRPFGSGSGTDFTLTISSLQAEDVAVYYC | (SEQ ID NO: 3) |
| FR4: | |
| FGQGQTKLEIK | (SEQ ID NO: 4) |

Exemplary heavy chain frameworks of human MAb 21/28′ CL have the following sequences:

| | |
|---|---|
| FR1: | |
| QVQLVQSGAEVKKPQASVKVSCKASQYTFT | (SEQ ID NO: 5) |
| FR2: | |
| WVRQAPGQRLEWMG | (SEQ ID NO: 6) |
| FR3: | |
| RVTITRDTSASTAYMELSSLRSEDTAVYYCAR | (SEQ ID NO: 7) |
| FR4: | |
| WGQGTLVTVSS. | (SEQ ID NO: 8) |

These framework sequences are provided for example only; a humanized antibody can include the human framework region from any human monoclonal antibody of interest.

Fragments of the HPC2 1-B3 antibody, a chimeric form thereof, or a humanized form thereof are also encompassed by the present disclosure. Antibodies, such as murine monoclonal antibodies, chimeric antibodies, and humanized antibodies, include full length molecules as well as fragments thereof, such as Fab, F(ab′)₂, and Fv, which include a heavy chain and light chain variable region and are capable of binding the epitope determinant. In some embodiments, the antibodies fragments have the sequences for V_{L} and V_{H} regions for the HPC2 1-B3 antibody. Fv antibodies are typically about 25 kDa and contain a complete antigen-binding site with three CDRs per each heavy chain and each light chain. To produce these antibodies, the V_{H} and the V_{L} can be expressed from two individual nucleic acid constructs in a host cell. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional example, the Fv fragments include V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which can be subsequently introduced into a host cell such as E. coli to recombinantly express the antibody fragment. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; Bird et al., Science 242:423, 1988; U.S. Pat. No. 4,946,778; and Pack et al., Bio/Technology 11:1271, 1993).

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys.89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

One of skill will realize that conservative variants of the antibodies can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules Amino acid substitutions (such as at most one, at most two, at most three, at most four, or at most five amino acid substitutions) can be made in the V_{H} and the V_{L} regions to increase yield. Conservative amino acid substitution tables providing functionally similar amino acids are well known to one of ordinary skill in the art. The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Thus, one of skill in the art can readily review the sequences of the HPC2 1-B3 antibody, locate one or more of the amino acids in the brief table above, identify a conservative substitution, and produce the conservative variant using well-known molecular biology techniques. Generally, conservative variants will bind the target antigen with an equal to or greater efficiency than the parent monoclonal antibody.

Effector molecules, such as therapeutic, diagnostic, or detection moieties can be linked to a HPC2 1-B3 antibody, a chimeric form thereof, or a humanized form thereof or a fragment thereof using any number of means known to those of skill in the art. Both covalent and noncovalent attachment means may be used. The procedure for attaching an effector molecule to an antibody varies according to the chemical structure of the effector. Polypeptides typically contain a variety of functional groups (such as carboxylic acid (COOH), free amine (—NH₂) or sulfhydryl (—SH) groups) which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company, Rockford, Ill. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site. When the target site is a tumor, a linker which is cleavable under conditions present at the tumor site (for example when exposed to tumor-associated enzymes or acidic pH) may be used.

In view of the large number of methods that have been reported for attaching a variety of radiodiagnostic compounds, radiotherapeutic compounds, labels (such as enzymes or fluorescent molecules), drugs, toxins, and other agents to antibodies one skilled in the art will be able to determine a suitable method for attaching a given agent to an antibody or other polypeptide.

In some examples a detectable label is linked to the HPC2 1-B3 antibody, a humanized form thereof or a fragment thereof. Detectable labels include but are not limited to fluorophores (for example FITC, PE and the like), enzymes (for example horseradish peroxidase, HRP), radiolabels, or electrodense particles, such as a nanoparticle (for example a gold particle or a semiconductor nanocrystal, such as a quantum dot (QDOT®).

In some examples a therapeutic agent is linked to the HPC2 1-B3 antibody, a chimeric form thereof, or a humanized form thereof or a fragment thereof. Therapeutic agents include various effector molecules such as drugs (for example, vinblastine, daunomycin and other chemotherapeutics), cytotoxins (for example native or modified Pseudomonas exotoxin or Diphtheria toxin). Therapeutic agents can include encapsulating agents (for example, liposomes) which themselves contain pharmacological compositions, target moieties and ligands. The choice of a particular therapeutic agent depends on the particular target molecule or cell and the biological effect desired to be evoked. Thus, for example, the therapeutic agent may be an effector molecule that is cytotoxic which is used to bring about the death of a particular target cell. Conversely, where it is merely desired to invoke a non-lethal biological response, a therapeutic agent can be conjugated to a non-lethal pharmacological agent or a liposome containing a non-lethal pharmacological agent.

Immunotoxins are chimeric molecules (such as a recombinant immunotoxins) in which a cell targeting moiety (for example an HPC2 1-B3 antibody, a chimeric form, a humanized form thereof or a fragment thereof) is fused to a toxin (see for example Pastan et al., Science, 254:1173-1177, 1991). If the cell targeting moiety is an antibody, the molecule can be termed a recombinant immunotoxin. The toxin moiety is typically genetically altered so that it cannot bind to the toxin receptor present on most normal cells. Thus, immunotoxins, such as recombinant immunotoxins, selectively kill cells which are recognized by the antigen binding domain.

Toxins can be employed with a HPC2 1-B3 antibody, a chimeric form thereof, or a humanized form thereof and fragments of these antibodies, such as a svFv or a dsFv, to yield chimeric molecules, which are of use as immunotoxins. Exemplary toxins include Pseudomonas exotoxin (PE), ricin, abrin, diphtheria toxin and subunits thereof, ribotoxin, ribonuclease, saporin, and calicheamicin, as well as botulinum toxins A through F. These toxins are well known in the art and many are readily available from commercial sources (for example, Sigma Chemical Company, St. Louis, Mo.).

Diphtheria toxin is isolated from Corynebacterium diphtheriae. Typically, diphtheria toxin for use in immunotoxins is mutated to reduce or to eliminate non-specific toxicity. A mutant known as CRM107, which has full enzymatic activity but markedly reduced non-specific toxicity, has been known since the 1970's (Laird and Groman, J. Virol. 19:220, 1976), and has been used in human clinical trials. See, U.S. Pat. No. 5,792,458 and U.S. Pat. No. 5,208,021. As used herein, the term “diphtheria toxin” refers as appropriate to native diphtheria toxin or to diphtheria toxin that retains enzymatic activity but which has been modified to reduce non-specific toxicity.

Ricin is the lectin RCA60 from Ricinus communis (Castor bean). The term “ricin” also references toxic variants thereof. For example, see, U.S. Pat. No. 5,079,163 and U.S. Pat. No. 4,689,401. Ricinus communis agglutinin (RCA) occurs in two forms designated RCA₆₀ and RCAₗ₂₀ according to their molecular weights of approximately 65 and 120 kD, respectively (Nicholson & Blaustein, J. Biochim. Biophys. Acta 266:543, 1972). The A chain is responsible for inactivating protein synthesis and killing cells. The B chain binds ricin to cell-surface galactose residues and facilitates transport of the A chain into the cytosol (Olsnes et al., Nature 249:627-631, 1974 and U.S. Pat. No. 3,060,165).

Ribonucleases have also been conjugated to targeting molecules for use as immunotoxins (see Suzuki et al., Nat. Biotech. 17:265-70, 1999). Exemplary ribotoxins such as α-sarcin and restrictocin are discussed in, for example, Rathore et al., Gene 190:31-5, 1997; and Goyal and Batra, Biochem 345 Pt 2:247-54, 2000. Calicheamicins were first isolated from Micromonospora echinospora and are members of the enediyne antitumor antibiotic family that cause double strand breaks in DNA that lead to apoptosis (see, for example, Lee et al., J. Antibiot 42:1070-87. 1989). The drug is the toxic moiety of an immunotoxin in clinical trials (see, for example, Gillespie et al., Ann Oncol 11:735-41, 2000).

Abrin includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B chain (abrin-b) binds to D-galactose residues (see, Funatsu et al., Agr. Biol. Chem. 52:1095, 1988; and Olsnes, Methods Enzymol. 50:330-335, 1978).

In one embodiment, the toxin is Pseudomonas exotoxin (PE). Native Pseudomonas exotoxin A (“PE”) is an extremely active monomeric protein (molecular weight 66 kD), secreted by Pseudomonas aeruginosa, which inhibits protein synthesis in eukaryotic cells. The native PE sequence and the sequence of modified PE are provided in U.S. Pat. No. 5,602,095. In one embodiment, native PE has a sequence set forth as:

(SEQ ID NO: 9)
AEEAFDLWNE CAKACVLDLK DGVRSSRMSV DPAIADTNGQ GVLHY
SMVLE GGNDALKLAI DNALSITSDG LTIRLEGGVE
PNKPVRYSYTRQ ARGSWSLN WLVPIGHEKP SNIKVFIHEL
NAGNQLSHMS PIYTIEMGDE LLAKLARDAT FFVRAHESNE
MQPTLAISHA GVSVVMAQTQ PRR EKR WSEW ASGKVLCLLD
PLDGVYNYLA QQRCNLDDTW EGKIYRVLAGN PAKHDLDIK
PTVISHRLHF PEGGSLAALT AHQACHLPLE TFTRHRQPRG
WEQLEQCGYP VQRLVALYLAARLSWNQVDQ VIRNALASPG
SGGDLGE AIR EQPEQARLAL TLAAAESERF VRQGTGNDEA
GAANADVVSL TCPVAA GECA GPADSGDALL ERNYPTGAEF
LGDGGDVSFS TRGTQNWTVERLLQ AHRQLE ERGYVFVGYH
GTFLEAAQSI VFGGVRARSQ DLDAIWRGFY IA GDPALAYG
YAQDQEPDAR GRIRNGALLR VYVPRSSLPG FYRTSLTLAAP
EAAGEVERL IGHPLPLRLD AITGPEEEGG
RLETILGWPLAERTVVIPSAIPTD PRNVGG DLDPSSIPDK
EQAISALPDYASQPGKPPRE DLK.

The method of action of PE is inactivation of the ADP-ribosylation of elongation factor 2 (EF-2). The exotoxin contains three structural domains that act in concert to cause cytotoxicity. Domain Ia (amino acids 1-252) mediates cell binding. Domain II (amino acids 253-364) is responsible for translocation into the cytosol and domain III (amino acids 400-613) mediates ADP ribosylation of elongation factor 2. The function of domain Ib (amino acids 365-399) remains undefined, although a large part of it, amino acids 365-380, can be deleted without loss of cytotoxicity. See Siegall et al., J. Biol. Chem. 264:14256-14261, 1989.

The term “Pseudomonas exotoxin” (“PE”) as used herein refers as appropriate to a full-length native (naturally occurring) PE or to a PE that has been modified. Such modifications may include, but are not limited to, elimination of domain Ia, various amino acid deletions in domains Ib, II and III, single amino acid substitutions and the addition of one or more sequences at the carboxyl terminus, such as KDEL (SEQ ID NO: 10) and REDL (SEQ ID NO: 11), see Siegall et al., supra. In several examples, the cytotoxic fragment of PE retains at least 50%, preferably 75%, more preferably at least 90%, and most preferably 95% of the cytotoxicity of native PE. In one embodiment, the cytotoxic fragment is more toxic than native PE.

Thus, the PE used in the immunotoxins disclosed herein includes the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell (for example as a protein or pre-protein). Cytotoxic fragments of PE known in the art include PE40, PE38, and PE35.

In several embodiments, the PE has been modified to reduce or eliminate non-specific cell binding, typically by deleting domain Ia, as taught in U.S. Pat. No. 4,892,827, although this can also be achieved, for example, by mutating certain residues of domain Ia. U.S. Pat. No. 5,512,658, for instance, discloses that a mutated PE in which Domain Ia is present but in which the basic residues of domain Ia at positions 57, 246, 247, and 249 are replaced with acidic residues (glutamic acid, or “E”) exhibits greatly diminished non-specific cytotoxicity. This mutant form of PE is sometimes referred to as PE4E.

PE40 is a truncated derivative of PE (see, Pai et al., Proc. Nat'l Acad. Sci. USA 88:3358-62, 1991; and Kondo et al., J. Biol. Chem. 263:9470-9475, 1988). PE35 is a 35 kD carboxyl-terminal fragment of PE in which amino acid residues 1-279 have been deleted and the molecule commences with a met at position 280 followed by amino acids 281-364 and 381-613 of native PE. PE35 and PE40 are disclosed, for example, in U.S. Pat. No. 5,602,095 and U.S. Pat. No. 4,892,827.

In some embodiments, the cytotoxic fragment PE38 is employed. PE38 is a truncated PE pro-protein composed of amino acids 253-364 and 381-613 of SEQ ID NO: 19 which is activated to its cytotoxic form upon processing within a cell (see for example, U.S. Pat. No. 5,608,039, and Pastan et al., Biochim. Biophys. Acta 1333:C1-C6, 1997).

While in some embodiments, the PE is PE4E, PE40, or PE38, any form of PE in which non-specific cytotoxicity has been eliminated or reduced to levels in which significant toxicity to non-targeted cells does not occur can be used in the immunotoxins disclosed herein so long as it remains capable of translocation and EF-2 ribosylation in a targeted cell.

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38.

The antibodies or antibody fragments disclosed herein can be derivatized or linked to another molecule (such as another peptide or protein). In general, the antibodies or portion thereof is derivatized such that the binding to the target is not affected adversely by the derivatization or labeling. For example, the antibody can be functionally linked (by chemical coupling, genetic fusion, noncovalent association or otherwise) to one or more other molecular entities, such as another antibody (for example, a bispecific antibody), a detection agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are available from Pierce Chemical Company, Rockford, Ill.

A HPC2 1-B3 antibody, a chimeric form thereof, a humanized form thereof or a fragment thereof can also be labeled with a detectable agent. Useful detectable agents include electron-dense compounds, enzymes, fluorochromes, a haptens, and radioisotopes. In some examples the disclosed antibodies are labeled with a fluorochrome, for example fluorescein, fluorescein isothiocyanate, rhodamine, 5-dimethylamine-1-napthalenesulfonyl chloride, phycoerythrin, lanthanide phosphors and the like. Bioluminescent markers are also of use, such as Green fluorescent protein (GFP), Yellow fluorescent protein (YFP) and enhanced variants of these proteins. The HPC2 1-B3 antibody can also be detected using secondary reagents with specificity for mouse IgG.

In other examples, a HPC2 1-B3 antibody, a chimeric form thereof, or a humanized form thereof or a fragment thereof is labeled with enzymes that are useful for detection, such as horseradish peroxidase, β-galactosidase, luciferase, alkaline phosphatase, glucose oxidase and the like. When an antibody is labeled with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody may also be labeled with biotin, and detected through indirect measurement of avidin or streptavidin binding. It should be noted that the avidin itself can be labeled with an enzyme or a fluorescent label.

A HPC2 1-B3 antibody, a chimeric form thereof, or a humanized form thereof or a fragment thereof can be labeled with a paramagnetic agent, such as gadolinium. Antibodies can also be labeled with lanthanides (such as europium and dysprosium), and manganese. Paramagnetic particles such as superparamagnetic iron oxide are also of use as labels. An antibody may also be labeled with a predetermined polypeptide epitopes recognized by a secondary reporter (such as leucine zipper pair sequences, binding sites for secondary antibodies, metal binding domains, epitope tags). In some embodiments, labels are attached by spacer arms of various lengths to reduce steric hindrance.

A HPC2 1-B3 antibody, a chimeric form thereof, or a humanized form thereof or a fragment thereof can also be labeled with a radiolabeled amino acid. The radiolabel may be used for both diagnostic and therapeutic purposes. For instance, the radiolabel may be used to detect pancreatic neoplasia cells for example by x-ray or other diagnostic techniques, such as positron emission tomography (PET) or magnetic resonance imaging (MRI).

Further, the radiolabel may be used therapeutically as a toxin for pancreatic adenocarcinoma. Examples of labels for antibodies include, but are not limited to: ³H, ¹⁴C, and ¹²⁵I.

A HPC2 1-B3 antibody, a chimeric form thereof, or a humanized form thereof or a fragment thereof can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

Nucleic acids encoding the amino acid sequences of the disclosed antibodies are also provided herein. Nucleic acids encoding antibodies produced by the hybridoma HPC2 1-B3 (or a chimeric or humanized form of any of these antibodies or fragment thereof) can readily be produced by one of skill in the art.

Nucleotides molecules encoding the antibodies can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same effector molecule or antibody sequence. Thus, nucleic acids encoding antibodies, conjugates and fusion proteins are provided herein.

Nucleic acid sequences encoding a HPC2 1-B3 antibody, a chimeric form thereof, or a humanized form thereof or a fragment thereof can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences may be obtained by the ligation of shorter sequences.

Exemplary nucleic acids encoding sequences encoding the HPC2 1-B3 antibody can be prepared by cloning techniques. Examples of appropriate cloning and sequencing techniques, and instructions sufficient to direct persons of skill through cloning are found in Sambrook et al., supra, Berger and Kimmel (eds.), supra, and Ausubel, supra. Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (San Diego, Calif.), and Applied Biosystems (Foster City, Calif.), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In one example, an antibody of use is prepared by inserting the cDNA which encodes a variable region from an antibody into a vector which comprises the cDNA encoding an effector molecule, such as an enzyme or label. The insertion is made so that the variable region and the effector molecule are read in frame so that one continuous polypeptide is produced. Thus, the encoded polypeptide contains a functional Fv region and a functional EM region. In one embodiment, cDNA encoding an enzyme is ligated to a scFv so that the enzyme is located at the carboxyl terminus of the scFv. In several examples, cDNA encoding a horseradish peroxidase or alkaline phosphatase, or a polypeptide marker of interest is ligated to a scFv so that the enzyme (or polypeptide marker) is located at the amino terminus of the scFv. In another example, the label is located at the amino terminus of the scFv. In a further example, cDNA encoding the protein or polypeptide marker is ligated to a heavy chain variable region of an antibody, so that the enzyme or polypeptide marker is located at the carboxyl terminus of the heavy chain variable region. The heavy chain-variable region can subsequently be ligated to a light chain variable region of the antibody using disulfide bonds. In a yet another example, cDNA encoding an enzyme or a polypeptide marker is ligated to a light chain variable region of an antibody, so that the enzyme or polypeptide marker is located at the carboxyl terminus of the light chain variable region. The light chain-variable region can subsequently be ligated to a heavy chain variable region of the antibody using disulfide bonds.

Once the nucleic acids encoding the immunotoxin, antibody, labeled antibody, or fragment thereof are isolated and cloned, the protein can be expressed in a recombinantly engineered cell such as bacteria, plant, yeast, insect and mammalian cells using a suitable expression vector. One or more DNA sequences encoding the antibody or fragment thereof can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding an antibody, labeled antibody, or functional fragment thereof, can be operatively linked to expression control sequences. An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The polynucleotide sequences encoding the antibody, labeled antibody, or functional fragment thereof can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding the antibody, labeled antibody, or functional fragment thereof, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression systems such as plasmids and vectors of use in producing proteins in cells including higher eukaryotic cells such as the COS, CHO, HeLa and myeloma cell lines.

Isolation and purification of recombinantly expressed polypeptide can be carried out by conventional means including preparative chromatography and immunological separations. Once expressed, the antibody, labeled antibody or functional fragment thereof can be purified according to standard procedures of the art, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are well-known and are applicable to the antibodies disclosed herein. See, Buchner et al., Anal. Biochem. 205:263-270, 1992; Pluckthun, Biotechnology 9:545, 1991; Huse et al., Science 246:1275, 1989 and Ward et al., Nature 341:544, 1989, all incorporated by reference herein.

Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry 9: 5015-5021, 1970, incorporated by reference herein, and especially as described by Buchner et al., supra.

Renaturation is typically accomplished by dilution (for example, 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. An exemplary yield is obtained when these two proteins are mixed in a molar ratio such that a 5 fold molar excess of one protein over the other is not exceeded. Excess oxidized glutathione or other oxidizing low molecular weight compounds can be added to the refolding solution after the redox-shuffling is completed.

In addition to recombinant methods, the antibodies, labeled antibodies and functional fragments thereof that are disclosed herein can also be constructed in whole or in part using standard peptide synthesis. Solid phase synthesis of the polypeptides of less than about 50 amino acids in length can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments. Methods of forming peptide bonds by activation of a carboxyl terminal end (such as by the use of the coupling reagent N, N′-dicylohexylcarbodimide) are well known in the art.

### B. Methods of Detection and Isolation

Methods are also provided for detecting and/or isolating a pancreatic neoplasm cell (such as an intraductal papillary mucinous neoplasm (IPMN) cell or a pancreatic adenocarcinoma cell, for example a pancreatic ductal adenocarcinoma cell), or an antigen secreted or otherwise liberated from a pancreatic neoplasm cell, for example from a biological sample. The method includes contacting the biological sample with the monoclonal HPC2 1-B3 antibody, a chimeric form thereof, or a humanized form thereof or a functional fragment thereof under conditions in which an immune complex will form between the HPC2 1-B3 antibody, chimeric form thereof, or humanized form thereof or functional fragment thereof and antigens that are specifically bound by HPC2 1-B3 antibody, chimeric form thereof, or humanized form thereof or functional fragment thereof sample, such as antigens present on the surface of a cell or even antigens that are not attached to a cell. In some examples, the presence (or absence) of the immune complex is then detected and/or used to isolate cells of interest. The presence of the immune complex indicates the presence of a neoplastic pancreatic cell, such as an IPMN cell or a pancreatic adenocarcinoma cell, for example a pancreatic ductal adenocarcinoma cell. In some examples, the methods are used to detect a pancreatic adenocarcinoma, such as a metastatic pancreatic adenocarcinoma. The method can be used to detect pancreatic cancer that has metastasized to a distal site, such as the liver. Thus, in one embodiment, and method is provided to detect metastatic pancreatic adenocarcinoma.

In some examples, the antigen is secreted or otherwise separated from the cancerous or precancerous pancreatic cell, such that the free antigen can be detected in a biological sample, such as a fluid sample, for example a serum sample obtained from the subject. In this way, the presence of antigen the specifically binds to the the HPC2 1-B3 antibody, a chimeric form thereof, or a humanized form thereof or a functional fragment thereof is used to detect antigen present in the biological sample and therefore detect the presence of the cancerous or precancerous pancreatic cell in the subject that the biological sample was obtained from.

In some embodiments a second antibody (such as an antibody that recognizes a mouse IgG) (which can be detectably labeled) that specifically binds the HPC2 1-B3 antibody, a chimeric form thereof, or a humanized form thereof or a functional fragment thereof is used to detect and/or isolate a neoplastic pancreatic cell from a sample. The sample can be any sample, including, but not limited to, tissue from biopsies, such as those obtained from autopsies and pathology specimens. Biological samples also include sections of tissues, such as frozen sections taken for histological purposes. Biological samples further include body fluids, for example cell free body fluid, such as duct fluid, blood, plasma, serum, tissue aspirate, spinal fluid or urine.

A biological sample is typically obtained from a mammal, such as a rat, mouse, cow, dog, guinea pig, rabbit, or primate, such as a human. Thus is some examples a subject, such as a human subject, is selected and a biological sample from that subject is tested for the presence of pancreatic cancer or a precancerous pancreatic lesion using the disclosed antibodies or fragments thereof. The subject can also be tested for the presence of metastatic pancreatic cancer, such as pancreatic cancer that has metastasized to the liver.

In some examples, the method is a method of diagnosing or confirming a diagnosis of a pancreatic adenocarcinoma, such as a ductal adenocarcinoma. IN additional examples, the methods are used to detect a pancreatic adenocarcinoma, such as a metastatic pancreatic adenocarcinoma. In some examples, the method is a method of diagnosing or confirming a diagnosis of a precancerous lesion, such as an IPMN, for example precancerous lesions classified as PanIN 1-3, such as PanIN 3. In some examples, the method is a method of grading a precancerous legion, for example grading a precancerous lesion as PanIN 1, PanIN 2 or PanIN 3. As PanIN 3 legions have a greater potential (whether realized or not) to develop into pancreatic carcinoma, such as pancreatic adenocarcinoma, the classification of a precancerous lesion as PanIN 3 can be used in the prognosis of a subject as one who will develop pancreatic carcinoma, such as pancreatic adenocarcinoma.

The method can include contacting a biological sample with the HPC2 1-B3 monoclonal antibody, chimeric form thereof, humanized form thereof, or functional fragment thereof under conditions wherein an immune complex will form and detecting the formation of the immune complex. In some embodiments, monoclonal antibody, chimeric form thereof, humanized form thereof, or functional fragment thereof is labeled. In other embodiments, the monoclonal antibody, chimeric form thereof, humanized form thereof, or functional fragment thereof is unlabeled. The method can also include contacting the biological sample with a second antibody that specifically binds the first monoclonal antibody, wherein the second antibody is labeled. The method can further include isolating the cell bound by the HPC2 1-B3 antibody, a chimeric form thereof, or a humanized form thereof or a functional fragment thereof.

In some examples the method is performed in vitro, for example when the biological sample is removed from the subject. In some examples the method is carried out in vivo for example by administering the disclosed antibodies, humanized form thereof or fragment thereof to a subject, such as a subject that has or is suspected of having a pancreatic neoplasia, such as a pancreatic tumor, for example a pancreatic adenocarcinoma.

The disclosed antibodies and fragments thereof can be used to monitor response to therapy. The number and or mass of pancreatic adenocarcinoma cells, such as the cells present in a subject, can be determined using the methods disclosed herein. In one embodiment, an increase in the number or mass of pancreatic adenocarcinoma cells, as compared to a control, such as the number or mass of pancreatic adenocarcinoma cells at an earlier time point, indicates that the pancreatic adenocarcinoma is progressing and that the therapy as not effective in reducing tumor burden. Conversely, a decrease in the number or mass of pancreatic adenocarcinoma cells, as compared to a control, such as the number or mass of pancreatic adenocarcinoma cells at an earlier time point, indicates that the pancreatic adenocarcinoma is regressing and that the therapy is effective. A control can be a standard value, or the number or mass of pancreatic adenocarcinoma cells in a sample from a subject not afflicted with a tumor or the number or mass of pancreatic adenocarcinoma cells in a sample from the subject at an earlier time point, for example prior to therapy.

The antibodies described herein can be used in immunohistochemical assays, such as on histological sections, including section of the pancreas and liver. These assays are well known to one of skill in the art (see Harlow & Lane, Antibodies, A Laboratory Manual, Cold Spring Harbor Publications, New York (1988), for a description of immunoassay formats). The antibodies can also be used for fluorescence activated cell sorting (FACS), for example to isolate a pancreatic neoplasm cell (such as an IPMN cell or a pancreatic adenocarcinoma cell, for example a pancreatic ductal adenocarcinoma cell). A FACS employs a plurality of color channels, low angle and obtuse light-scattering detection channels, and impedance channels, among other more sophisticated levels of detection, to separate or sort cells (see U.S. Pat. No. 5,061,620). The antibodies can also be used for magnetic separation of pancreatic neoplasm cells (such as IPMN cells or pancreatic adenocarcinoma cells, for example pancreatic ductal adenocarcinoma cells). Magnetic separation involves the use of paramagnetic particles which are 1) conjugated to the pancreatic neoplasm specific antibody HPC2 1-B3, a chimeric form thereof, or a humanized form thereof or a fragment thereof; 2) conjugated to detection antibodies which are able to bind to the HPC2 1-B3 antibodies, a chimeric form thereof, or a humanized form thereof or a fragment thereof; or 3) conjugated to a detection reagent (such as avidin) which can bind to detection antibodies (such as biotinylated antibodies). Any of the antibodies disclosed herein can be used in these assays. The antibodies can be used in methods that utilize positive selection (expressing the antigen of interest), negative selection (not expressing the antigen of interest), or both (expressing one antigen of interest and not expressing a second antigen of interest).

The HPC2 1-B3 antibody, a chimeric form thereof, or a humanized form thereof or a fragment thereof disclosed herein can also be used to detect pancreatic neoplasm cells, such as an IPMN cells or a pancreatic adenocarcinoma cells, for example a pancreatic ductal adenocarcinoma cell, including metastatic cells, in vivo. The antibodies disclosed herein can also be used to detect pancreatic tumors, such as pancreatic adenocarcinoma, for example pancreatic ductal adenocarcinoma in vivo. In one embodiment, a HPC2 1-B3 antibody, a chimeric form thereof, or a humanized form thereof or a fragment thereof is administered to the subject for a sufficient amount of time for the antibody to localize to the pancreas (or tumor) in the subject and to form an immune complex with the pancreatic cells (or tumor). The immune complex can then be detected for example radiolocalization, radioimaging, MRI, PET scan, or fluorescence imaging, for example by using a detectibly labeled antibody, humanized form thereof or functional fragment thereof. Once detected, in an ectopic location (as in a tumor) the test results can be used to assist in or guide surgical or other excision of a tumor.

In vivo imaging methods can also be utilized with the antibodies disclosed herein. These technologies include magnetic resonance imaging (for example using a biotinylated antibody and avidin-iron oxide), positron emission tomography (for example using an ¹¹¹indium-labeled monoclonal antibody), and optical imaging (for example using luciferase or green fluorescent protein labeled antibodies). In one example, magnetic resonance imaging is utilized. In the setting of magnetic resonance imaging, contrast agent detection can be greatly impacted by magnetic resonance scanner field strength. Increased field strengths provide improvements by orders of magnitude in the ability to detect contrast agents (Hu et al., Annu Rev Biomed Eng. 6:157-184, 2004; Wedeking et al., Magn. Reson. Imaging. 17:569-575, 1999). For example, the limit of detection of gadolinium at 2 tesla (T) is ˜30 μM. At 4T the limit of detection is reduced to ˜1 μM. With newly available 7 to 12T scanners one would expect to detect low (10-100) nM concentrations of this contrast agent. Similar sensitivity can also be identified using contrast agents such as iron oxide.

### C. Pharmaceutical Compositions and Therapeutic Methods

Pharmaceutical compositions are disclosed herein that include a HPC2 1-B3 antibody, a chimeric form thereof, or a humanized form thereof or a fragment thereof. These pharmaceutical compositions are for use in methods of treatment and/or methods of detection, and can be formulated with an appropriate solid or liquid carrier, depending upon the particular mode of administration chosen. In addition, a monoclonal antibody linked to an effector molecule (i.e., toxin, chemotherapeutic drug, or detectable label) can be prepared in pharmaceutical compositions. Compositions including an antibody that specifically binds pancreatic neoplasm cells or a subset thereof are of use, for example, for the treatment of pancreatic neoplasia, such as IPMN or a pancreatic adenocarcinoma for example a pancreatic ductal adenocarcinoma cell.

The pharmaceutically acceptable carriers and excipients useful in this disclosure, for either therapeutic or diagnostic methods, are conventional. For instance, parenteral formulations usually comprise injectable fluids that are pharmaceutically and physiologically acceptable fluid vehicles such as water, physiological saline, other balanced salt solutions, aqueous dextrose, glycerol or the like. Excipients that can be included are, for instance, other proteins, such as human serum albumin or plasma preparations. If desired, the pharmaceutical composition to be administered can also contain minor amounts of non-toxic auxiliary substances, such as wetting or emulsifying agents, preservatives, and pH buffering agents and the like, for example sodium acetate or sorbitan monolaurate.

The dosage form of the pharmaceutical composition will be determined by the mode of administration chosen. For instance, in addition to injectable fluids, topical, inhalation, oral and suppository formulations can be employed. Topical preparations can include ointments, sprays and the like. Inhalation preparations can be liquid (such as solutions or suspensions) and include mists, sprays and the like. Oral formulations can be liquid (for example, syrups, solutions or suspensions), or solid (such as powders, pills, tablets, or capsules). Suppository preparations can also be solid, gel, or in a suspension form. For solid compositions, conventional non-toxic solid carriers can include pharmaceutical grades of mannitol, lactose, starch, or magnesium stearate. Actual methods of preparing such dosage forms are known, or will be apparent, to those skilled in the art.

The pharmaceutical compositions that include a HPC2 1-B3 antibody, a chimeric form thereof, or a humanized form thereof or a fragment thereof can be formulated in unit dosage form suitable for individual administration of precise dosages. In addition, the pharmaceutical compositions may be administered in a single dose or as in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regime will also, at least in part, be determined based on the particular needs of the subject to be treated and will be dependent upon the judgment of the administering practitioner. In one specific, non-limiting example, a unit dosage can be about 0.1 to about 10 mg per patient per day. Dosages from about 0.1 up to about 100 mg per patient per day may be used, particularly if the agent is administered to a secluded site and not into the circulatory or lymph system, such as into a body cavity, into a lumen of an organ, or directly into a tumor. In one embodiment, about 10 mCi of a radiolabeled monoclonal antibody is administered to a subject. In other embodiments, about 15 mCi, about 20 mCi, about 50 mCi, about 75 mCi or about 100 mCi of a radiolabeled monoclonal antibody is administered to a subject. The amount of active compound(s) administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated.

The compounds of this disclosure can be administered to humans on whose tissues they are effective in various manners such as administration into the tumor. However, administration topically, orally, intravascularly such as intravenously, intramuscularly, intraperitoneally, intranasally, intradermally, intrathecally, subcutaneously, via inhalation or via suppository is of use with the antibodies disclosed herein. The particular mode of administration and the dosage regimen will be selected by the attending clinician, taking into account the particulars of the case (for example the subject, the disease, the disease state involved, and whether the treatment is prophylactic).

Methods for inhibiting the growth a pancreatic neoplasm cell (such as an IPMN cell or a pancreatic adenocarcinoma cell, for example a pancreatic ductal adenocarcinoma cell) are disclosed. The methods include contacting the cell with an effective amount of a HPC2 1-B3 antibody, a chimeric form thereof, or a humanized form thereof or a fragment thereof conjugated to an effector molecule. The HPC2 1-B3 antibody, a chimeric form thereof, or a humanized form thereof or a fragment thereof disclosed herein can be used to target a therapeutic agent to pancreatic neoplasia cells, such as IPMN cells or pancreatic adenocarcinoma cells, for example pancreatic ductal adenocarcinoma cells. Treating pancreatic cells (as in a tumor) in a subject includes the administration of a HPC2 1-B3 antibody, a chimeric form thereof, or a humanized form thereof or a fragment thereof complexed to an effector molecule, such as, but not limited to, a radioactive isotope or other chemotherapeutic agent. In one embodiment, the antibody is complexed to an effector molecule, such as a radioactive isotope, is administered to a subject prior to surgery or treatment. In another embodiment, the antibody complexed to an effector molecule, such as a radioactive isotope, is administered to a subject following surgery or treatment. In additional examples, an antibody that specifically binds pancreatic adenocarcinoma cells can be administered to a subject prior to, or following, treatment for a pancreatic adenocarcinoma. Thus, the effectiveness of the treatment can be assessed.

In one embodiment, a therapeutically effective amount of an antibody is the amount necessary to inhibit further growth of a pancreatic adenocarcinoma, or the amount that is effective at reducing a sign or a symptom of the tumor, or reducing metatstasis. In another embodiment, a therapeutically effective amount of an antibody is the amount sufficient to visualize a pancreatic neoplasm cells, such as an IPMN cell or a pancreatic adenocarcinoma cell, for example a pancreatic ductal adenocarcinoma cell. It is advantageous for this dose to be administered in a human subject without eliciting a human anti-mouse (HAMA) response in the subject receiving the treatment.

Controlled release parenteral formulations of a monoclonal antibody can be made as implants, oily injections, or as particulate systems. For a broad overview of protein delivery systems (see Banga, A. J., Therapeutic Peptides and Proteins: Formulation, Processing, and Delivery Systems, Technomic Publishing Company, Inc., Lancaster, Pa., 1995). Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, J., Colloidal Drug Delivery Systems, J. Kreuter, ed., Marcel Dekker, Inc., New York, N.Y., pp. 219-342, 1994; Tice & Tabibi, Treatise on Controlled Drug Delivery, A. Kydonieus, ed., Marcel Dekker, Inc. New York, N.Y., pp. 315-339, 1992).

Polymers can be used for ion-controlled release. Various degradable and nondegradable polymeric matrices for use in controlled drug delivery are known in the art (Langer, R., Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec et al., J. Parent. Sci. Tech. 44:58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri, et al., Liposome Drug Delivery Systems, Technomic Publishing Co., Inc., Lancaster, Pa., 1993). Numerous additional systems for controlled delivery of therapeutic proteins are known (see, for example, U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496).

Site-specific administration of the disclosed compounds can be used, for instance by applying the antibody a region of tissue from which a tumor has been removed, or a region suspected of being prone to tumor development. In some embodiments, sustained intra-tumoral (or near-tumoral) release of the pharmaceutical preparation that includes a therapeutically effective amount of the antibody may be beneficial.

The present disclosure also includes therapeutic uses of monoclonal antibodies that are non-covalently or covalently linked to effector molecules. In one specific embodiment, the monoclonal antibody is covalently linked to an effector molecule that is toxic to a pancreatic tumor. In one specific, non-limiting example, the effector molecule is a cytotoxin. In other specific, non-limiting examples the effector molecule is a radioactive isotope, a chemotherapeutic drug, a bacterially-expressed toxin, a virally-expressed toxin, a venom protein, or a cytokine. Monoclonal antibodies covalently linked to an effector molecule have a variety of uses. For example, an antibody linked to a radioactive isotope is of use in immunotherapy. An antibody covalently linked to a radioactive isotope is of use to localize a tumor in radioimmunoguided surgery, such that the tumor can be removed.

The present disclosure also includes combinations of a monoclonal antibody, with one or more other agents useful in the treatment of tumors. For example, the compounds of this disclosure can be administered in combination with effective doses of immunostimulants, anti-cancer agents (such as chemotherapeutics), anti-inflammatory agents, anti-infectives, and/or vaccines. The term “administration in combination” or “co-administration” refers to both concurrent and sequential administration of the active agents.

### D. Kits

Kits are also provided herein. Kits for detecting and/or treating a pancreatic neoplasm cell contain a HPC2 1-B3 antibody, a chimeric form thereof, or a humanized form thereof or a fragment thereof. In some embodiments, an antibody fragment, such as an Fv fragment is included in the kit. In one example, such as for in vivo uses, the antibody can be a scFv fragment. In a further embodiment, the antibody is labeled (for example, with a fluorescent, radioactive, or an enzymatic label).

In one embodiment, a kit includes instructional materials disclosing means of use of an antibody that specifically binds pancreatic cells. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting pancreatic neoplasia cells in a biological sample generally includes the steps of contacting the biological sample with an antibody which specifically reacts, under immunologically reactive conditions, to antigen on the pancreatic cells of interest or secreted or otherwise liberated from the pancreatic cells of interest. The antibody is allowed to specifically bind under immunologically reactive conditions to form an immune complex, and the presence of the immune complex (bound antibody) is detected directly or indirectly.

Methods of determining the presence or absence of a cell surface antigen secreted or otherwise liberated antigen are well known in the art. For example, the antibodies can be conjugated to other compounds including, but not limited to, enzymes, paramagnetic beads, colloidal magnetic beads, haptens, fluorochromes, metal compounds, radioactive compounds or drugs, as described herein. The antibodies can also be utilized in immunoassays such as but not limited to radioimmunoassays (RIAs), enzyme linked immunosorbant assays (ELISA), Western blot analyses, or immunohistochemical assays.

The disclosure is illustrated by the following non-limiting Examples.

### EXAMPLES

### Example 1

### Production of Monoclonal Antibodies

This example describes the generation of a hybridoma secreting the HPC2 1-B3 antibody.

For the generation of these antibodies, BALB/c mice were immunized with enzyme-dispersed fresh human pancreatic adenocarcinoma cells Animals were immunized three times and the spleens were harvested. Four days after the final boosts, splenocyes were fused with SP2/0 myeloma cells. 600-800 hybridoma clones were picked after 10-14 days of growth and plated in methylcellulose media and subcultured in 96-well plates. Supernatants from fused cells were screened for desirable antibodies on frozen sections of pancreatic adenocarcinoma and normal pancreas. Stained sections were analyzed by fluorescence microscopy for novel antibodies reacting with pancreatic adenocarcinoma cells. Section bound primary antibodies were detected using a polyclonal Cy3-conjugated anti-mouse secondary antibody, and Cy3 was visualized by fluorescence microscopy. Monoclonal antibodies found to selectively react with pancreatic adenocarcinoma cells were further characterized. HPC2 1-B3 was one of the monoclonal antibodies found to react with pancreatic adenocarcinoma and not with normal pancreas.

### Example 2

### Characterization of the HPC2 1-B3 Monoclonal Antibody Staining of Pancreatic Tissue

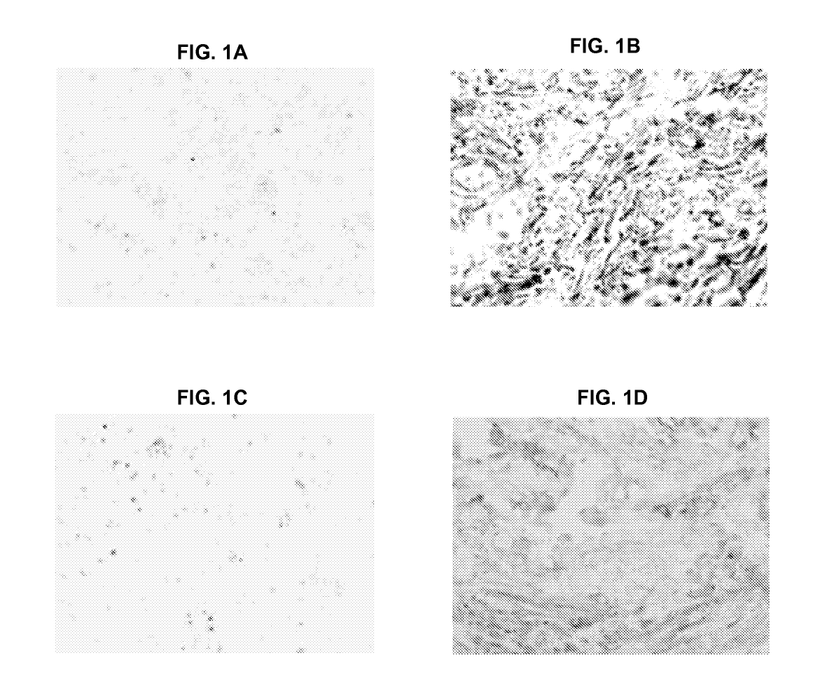

This example describes the evaluation of the HPC2 1-B3 monoclonal antibody for specific binding to normal tissue, on specimens of pancreatitis, and on specimens of pancreatic adenocarcinoma. The HPC2 1-B3 antigen has been detected in 16 of 17 pancreatic adenocarcinoma specimens (it failed to react with 1 poorly differentiated cancer), it was found on 0 of 9 specimens of normal pancreas, and it has been found on 0 of 6 specimens of pancreatitis (see FIG. 1). Thus, HPC2 1-B3 effectively distinguishes between pancreatic adenocarcinoma and both pancreatitis and normal pancreas.

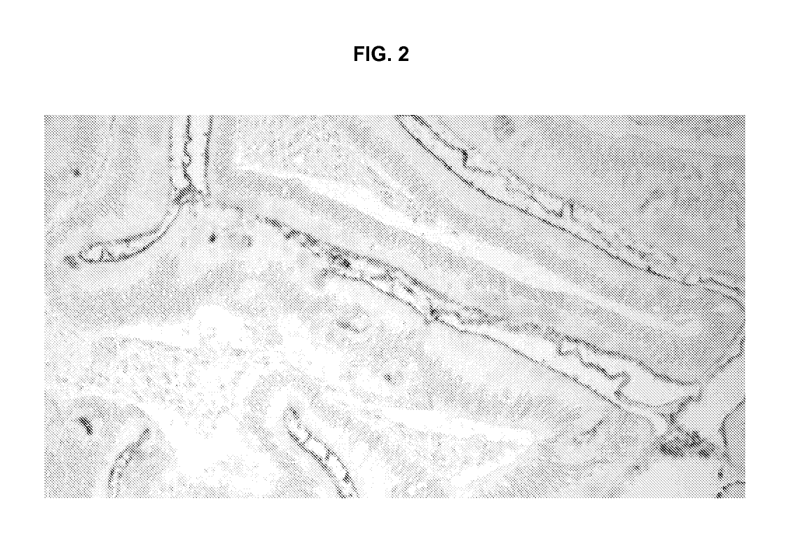

The HPC2 1-B3 antigen was also observed in specimens of intraductal papillary mucinous neoplasia (IPMN). IPMN is classified into three grades of pancreatic intraepithelial neoplasia (PanIN 1-3): Grade 1 (PanIN 1) is low grade, resembling normal pancreatic duct nuclei. Grade 2 (PanIN 2) has more nuclear atypia, and grade 3 (PanIN 3) has high grade nuclei, but is not invasive, which distinguishes PanIN 3 from adenocarcinoma Immunostaining of PanIN 1-3 with HPC2 1-B3 has revealed extensive reactivity with IPMN: The HPC2 1-B3 antigen was detected in 100% of PanIN 3 cases (4 or 4); 50% of PanIN 2 cases (1 of 2); and 45% of PanIN 1 cases (5 of 11). An example of HPC2 1-B3 staining of PanIN 1 is shown in FIG. 2. This figure illustrates expression of the HPC2 1-B3 antigen on the luminal aspect of the duct, and suggests that the HPC2 1-B3 antigen is secreted.

The observation that the HPC2 1-B3 antigen is expressed on IPMN specimens distinguishes this marker from other reported pancreatic cancer associated markers, suggesting utility of for this marker in early diagnosis of this disease. In addition, there is data suggesting that low grade PanIN 1-2 is less likely to progress to cancer than high grade PanIN-3. Therefore, the absence of HPC2 1-B3 staining may suggest the IPMN is low grade and may be followed, rather than warranting the high morbidity and mortality associated with Whipple surgical resection of the pancreas.

The recently identified KOC marker was analyzed in the PanIN specimens found to be reactive with the HPC2 1-B3 antibody. KOC, is an RNA-binding protein that is highly expressed in pancreatic adenocarcinoma and is not present in normal pancreas. Expression of the KOC and the HPC2 1-B3 antigens in these tissues was found to be markedly different, with the HPC2 1-B3 antigen identified at high frequency, particularly in PanIN 3, and the KOC antigen not found in PanIN 1-3 (Table 1). Thus, these data suggest that the HPC2 1-B3 antigen may be a better target than the KOC antigen in developing screening assays for detection of early stage pancreatic adenocarcinoma and precancerous lesions of the pancreas.

TABLE 1
The HPC2 1-B3 Antigen is Expressed in Precancerous
Lesions of the Pancreas and the KOC Antigen is not.*
Percent of cases where marker was detected.

| Antigen | PanIN 1 (11 cases) | PanIN 2 (2 cases) | PanIN 3 (4 cases) |
|---|---|---|---|
| HPC2 1-B3 | 45% | 50% | 100% |
| KOC | O % | 0% | 0% |

### Evaluation of HPC2 1-B3 Antigen Exrpesion by Tissue Microarray

Expression of the HPC2 1-B3 antigen has also been assessed by tissue microarray in approximately 50 different normal and cancer tissues. While most of these tissues were found to be negative for the HPC2 1-B3 antigen (Table 2), antigen expression was observed on cell subsets in normal lung and salivary gland, as well as in cervical squamous cell carcinoma and nasopharyngeal cancer.

TABLE 2
Evaluated Tissues where the HPC2 1-B3
Antigen has NOT been Observed.

| Normal Tissue | Cancer |
|---|---|
| Adrenal | Adrenal cortical adenoma |
| Bladder | Astrocytoma |
| Brain | B-cell non-Hodgkin's lymphoma |
| Cardiac muscle | Breast cancer |
| Colon | Carcinoid tumor |
| Endometrium | Clear cell carcinoma (renal) |
| Kidney | Colon adenocarcinoma |
| Liver | Esophageal squamous cell carcinoma |
| Lymph node | Hepatocellular carcinoma |
| Myometrium | Hodgkin's lymphoma |
| Pancreas | Lung squamous cell carcinoma |
| Placenta | Lung adenocarcinoma |
| Prostate | Lyomyosarcoma |
| Skeletal muscle | Meningioma |
| Small intestine | Ovarian cancer |
| Smooth muscle | Prostate cancer |
| Squamous mucosa | Rectal adenocarcinoma |
| Testis | Salivary gland adenoma |
| Thyroid | Sarcoma |
| | Small cell lung cancer |
| | Small intestine adenocarcinoma |
| | Stomach adenocarcinoma |
| | T-cell non-Hodgkin's lymphoma |
| | Transition cell carcinoma |

HPC2 1-B3 was also found to react similarly on formalin-fixed, paraffin-embedded, tissue. This observation is important as paraffin-embedding of tissues is the standard for assessment of pathology. The observation that HPC2 1-B3 reacts with paraffin-embedded tissue created the opportunity for rapid assessment of many tissues using tissue microarrays.

Evaluation of HPC2 1-B3 Antigen Exrpesion by flow cytometry

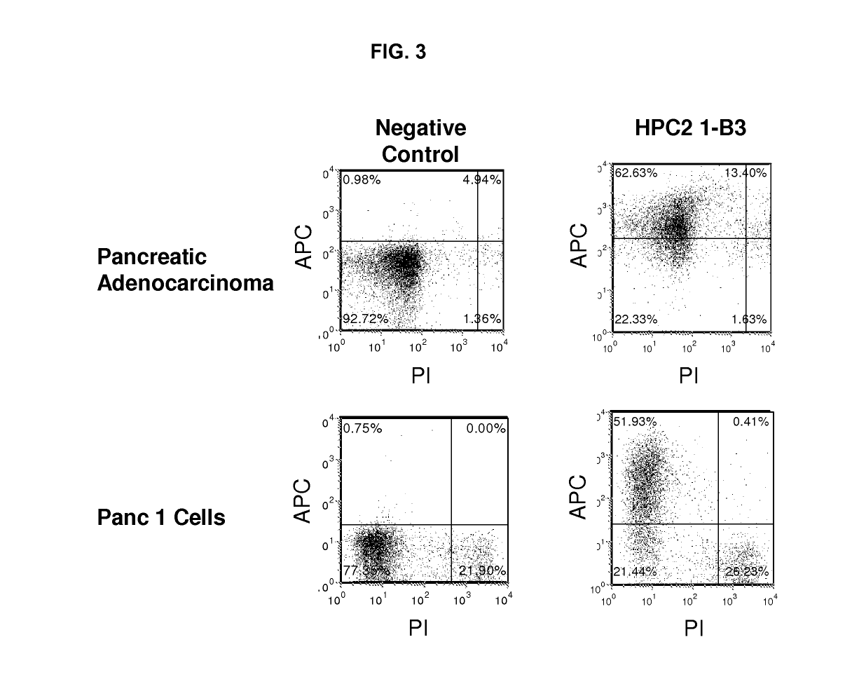

The HPC2 1-B3 antigen has also been found by flow cytometry to react with a cell surface antigen on Panc1 cells, a human pancreatic cancer cell line, and specimens of human pancreatic adenocarcinoma (see FIG. 3). Flow cytometry was performed using HPC2 1-B3 as the primary antibody and an anti-mouse APC conjugated antibody for the secondary antibody. This finding is consistent with the antigen distribution illustrated on IPMN in FIG. 2.

### Example 3

### Humanization and Production of an scFv

The CDR amino acid sequences of the monoclonal antibody HPC2 1-B3 antibody are determined. CDR amino acid sequences from monoclonal antibody HPC2 1-B3 are used for humanization and construction of recombinant scFv and scFv₂ fragments. Protein-based and cell-based assays have been used extensively for the purpose of evaluating engineered antibodies (reviewed by Qu et al, Methods. 36:84-95, 2005).

Competitive cell-based binding assays are developed to compare the antigen binding capabilities of engineered antibodies with those of the parental mouse monoclonal antibodies. For initial assays, trypsin-dispersed (0.05% for 5-10 minutes at 37° C.) human pancreatic adenocarcinoma cells are used as a source of target cells. For these assays, unlabeled engineered antibodies are used as a competitor of antigen binding by phycoerythrin (PE)-labeled parental antibodies. Briefly, dispersed pancreatic adenocarcinoma cells are plated at 1×10⁵ cells/well in a 96-well plate (100 uL/well). A constant amount of PE-labeled parental antibody (10 nM) is mixed with varying concentrations of unlabeled parental or engineered antibodies (0.2-1,000 nM) and added to each well (100 uL/well), with each experimental condition set up in triplicate. Plates are preblocked to prevent binding of PE-conjugated antibody to the plate surface (phosphate buffered saline (PBS), 0.05% Tween 20, and 5% fetal calf serum (FCS) for 2 hours at room temperature). After adding cells plus antibodies, the plates are incubated on ice with gentle mixing for 2 hr. Plates are then centrifuged and washed five times to eliminate unbound PE-labeled antibody and evaluated for PE signal using a fluorescence plate reader. The fluorescence associated with cells is plotted versus the concentration of unlabeled antibodies, yielding competitive inhibition curves. Successful engineering results in similar curves for the engineered and parental antibodies. Competitive radio-immunoassays can also be used as an alternative for this determination.

### Example 4

### Identification of the Antigen Bound by the HPC2 1-B3 Monoclonal Antibody by Expression Cloning

This example describes expression cloning to identify proteins recognized by monoclonal antibody HPC2 1-B3. A pancreatic adenocarcinoma cDNA library containing all possible antigen-encoding genes in pVPack packing vector constructs is combined with plasmids encoding retroviral structural proteins and a broad-tropism retroviral envelope (VSV-G) for the co-transfection of 293T cells to produce a retroviral library. Once produced, this library is used to stably transduce a suitable human target cell line, such as Paca2 or HUH7. Transduced target cells are then incubated with the monoclonal antibody HPC2 1-B3. Bound primary antibody is detected using an APC-conjugated secondary antibody, and positively labeled cells will be isolated using the inFlux™ cell sorter (Cytopeia/Becton Dickinson). Since the transduced cells contain a retroviral integrant which is not lost upon replication, cells will be sorted, expanded in culture (to increase absolute number of antigen expressing cells), and resorted (to ensure purity and to simplify genomic DNA preparation). The identity of the gene coding for a particular antigen will be determined by DNA sequencing using a primer binding to the promoter region of the pVPack construct.

In some examples candidate antigen gene sequences are identified using GenBank® and those sequences are used to design primers for extracellular domain sequence amplification. Type I proteins are expressed as human IgG1 Fc constructs and Type II proteins are expressed with an N-terminal Flag tag. Fusion proteins are detected and purified by their tags.

In some example the candidate antigen gene sequences of proteins of interest are amplified by PCR from cDNA and cloned into a pCR-3 mammalian expression vector (Invitrogen®) modified to include the following features: 1) a multiple cloning site; and 2) a cassette encoding the hinge, CH2 and CH3 domains of human IgG1 (GENBANK® accession number X70421, as available on Apr. 17, 2006).

In some examples cDNA are amplified with a high fidelity DNA polymerase, and the PCR product is cloned into a pCR-blunt vector for sequencing. The cDNA is recloned into the appropriate PCR-3 modified expression vectors and used for either transient expression using 293T cells or stable expression by HEK293 (ATCC CRL 1573). Fc-chimeric proteins are purified using a protein A column (Pierce), while Flag-tag recombinant protein are purified on an M2-agarose (Sigma) column.

### Example 5

### Identification of the Antigen Bound by the HPC2 1-B3 Monoclonal Antibody by Proteomic Analyses and Additional Methods

This example describes proteomic analyses to identify proteins recognized by monoclonal antibody HPC2 1-B3 Immunoaffinity column chromatography is used to isolate/enrich the target protein. Briefly, HPC2 1-B3 monoclonal antibodies at concentrations of 1-5 mg/mL are covalently coupled to an agarose matrix (AminoLink gel; Pierce; as per the manufacturer's instructions). This material is then used as the affinity matrix for column-based immunoaffinity purification of antigen. For antigen purification, cell or tissue lysates containing target antigens are loaded onto these columns at neutral pH, columns are washed to eliminate contaminating proteins, and target antigens are eluted by adjusting the buffer pH to ˜2.8. Elution fractions containing target antigen are identified by dot blot analyses.

Column enriched protein antigens are run on 1D SDS PAGE gels, and bands corresponding to immunoreactive species (determined by Western Blot of duplicate gels) are excised, subjected to tryptic digestion, and analyzed by nanoLC/MS/MS. Briefly, gel slices are washed to remove coomassie stain and then dehydrated by the addition of neat acetonitrile (ACN). Gel slices are treated with DTT and iodoacetamide to reduce and alkylate cystines, and prior to proteolysis, the gel slices will be washed and dried again. Proteolysis with trypsin is carried out overnight at 37° C. Peptides are extracted from the gel slices by the addition of two aliquots of 1% formic acid.

Protein identification and quantification is carried out using an Applied Biosystems Qstar XL. Briefly, 5 uL of peptides from the digest will be injected onto a reverse phase trap column, washed thoroughly, and then switched in-line with a 15 cm×75 uM analytical column packed with C18 reverse phase material. Peptides are eluted with increasing percent of organic gradient (0-40% ACN) and introduced to the mass spectrometer via an electrospray interface. Data dependent acquisition is used to select precursor ions and set collision energy for collisionally induced dissociation (CID) of the three most abundant ions derived from each survey scan. Product ion spectra is used to obtain protein identification via database searching using the MASCOT® (Matrix science) search engine.

### Example 6

### Detection of the Antigen Recognized by the HPC2 1-B3 Monoclonal Antibody in Body Fluids

This example describes a strategy to detect the antigen recognized by the HPC2 1-B3 monoclonal antibody in body fluids. Detection of the antigen in body fluids may facilitate disease diagnosis in patients not previously diagnosed with disease, confirmation of diagnosis in patients with tentative diagnosis, or disease monitoring in patients undergoing treatment for disease.

Plasma, serium, pancreatic tissue aspirates, and pancreatic duct fluid specimens are evaluated from patients with pancreatic adenocarcinoma for the presence of tumor antigen (for example the tumor antigen identified using the methods in Example 4) bound by monoclonal antibody HPC2 1-B3. For this series of tests, these fluid specimens are evaluated from 20-30 pancreatic cancer patients using Western blot analyses, direct ELISA, or sandwich ELISA. Antibody reactivity with serum specimens from normal donors (negative controls) is also assessed. Duct fluid from organ donors (from normal donors and from donors with pancreatitis) can also be assessed. The fluid is a negative duct fluid control. Blood samples (10 mL) for preparation of serum is obtained and serum is aliquoted and stored at −80° C. until used. Pancreatic ductal fluid is obtained from cancer patients during pancreatic resection for cancer. This is mixed with a complete mini-protease inhibitor (Roche), aliquoted and stored at −80° C. until used in assays identified above.

### Example 7

### Identification of Circulating Cells Expressing the Antigen Recognized by the HPC2 1-B3 Monoclonal Antibody

There is a growing body of literature on the detection of cancer cells in peripheral blood (see for example, Hayes et al., Clin Cancer Res.12:4218-24, 2006). The detection of circulating cancer cells can be used for the assessment of tumor cell tumorigenic potential or the responsiveness of particular cancers to candidate therapeutic agents. As the antibodies described herein have been shown to react with cell surface molecules, they are ideally suited for assessment and sorting of circulating live tumor cells from patients with pancreatic adenocarcinoma. Circulating cancer cells are present at low frequencies, ranging from 1 in 10³ to 1 in 10⁷ white blood cells. To determine whether pancreatic ductal adenocarcinoma cells are present in peripheral blood, flow cytometry and RT-PCR can be utilized. The detection, quantitation, and isolation of rare cells from peripheral blood typically uses a multi-step preparative process. Briefly, blood is collected in tubes containing an anti-coagulant (lithium heparin or sodium citrate). Red blood cells are lysed using a red blood cell lysis buffer (eBiosciences). Ficoll is not used, as it may result in loss of rare cells in the red blood cell pellet. Cells remaining following red blood cell lysis are stained with a Fluorescein Isothyocyanate (FITC)-conjugated a detectably labeled antibody (such as Fluorescein Isothyocyanate (FITC)-conjugated antibody) directed against CD45 (a marker expressed on all hematopoietic cells and not on ductal adenocarcinoma cells), with detectably labeled HPC2 1-B3 antibodies (such as R-Phycoerythrin (PE)-conjugated HPC2 1-B3 antibodies) directed against ductal adenocarcinoma cells (to detect cancer cells), and with propidium iodide (PI; a DNA-binding dye that reacts with dead cells).

Using (FITC)-conjugated CD45 antibody and PE-conjugated HPC2 1-B3 antibody, when the cells are analyzed or sorted, most cells are excluded from the analysis as they are PI positive (dead) or FITC positive (hematopoietic cells). Cells that do not stain with PI or FITC are assessed for the presence of PE. Cells that are PI negative, FITC negative, and PE positive are sorted as candidate circulating cancer cells. A cancer origin of the cells is confirmed using RT-PCR, where sorted cells are evaluated for the presence of the antigen bound by the HPC2 1-B3 monoclonal antibody, for example as identified in Examples 3 or 4. Controls can include isotype control antibodies and peripheral blood from normal donors.

### Example 8

### The Monoclonal Antibody, HPC2 1-B3, Distinguishes Pancreatic Adenocarcinoma from Cholangiocarcinoma and Gallbladder Cancer

This example describes that HPC2 1-B3 can be used to distinguish primary cholangiocarcinoma, gallbladder cancer, and metastatic colon cancer, from metastatic pancreatic or ampullary carcinoma in liver.

The monoclonal antibody, HPC2 1-B3, was found by immunohistochemical methods to selectively react with pancreatic ductal cancer and precancerous pancreatic lesions (PanINs), and it does not react with cells in normal pancreas or with cells in inflamed pancreas (pancreatitis). It was also investigated if HPC2 1-B3 would have application in early diagnosis of pancreatic cancer and in disease monitoring. The objective of this study was to determine if HPC2 1-B3 can distinguish metastatic pancreatic ductal cancer from other carcinomas, including cholangiocarcinoma, gallbladder cancer, ampullary cancer and metastatic colon cancer. A primary focus was to determine if HPC2 1-B3 could distinguish metastatic pancreatic cancer localized in the liver from other cancers present in liver.

To date, 50 cases have been investigated. Metastatic pancreatic carcinoma (n=17), cholangiocarcinoma in liver (n=10), primary gallbladder cancer (n=3), primary and metastatic ampullary carcinoma (n=10), and metastatic colon cancer to liver (n=10) were retrospectively identified from surgical pathology archives dated 2001-2009. Histologic sections were immunostained for HPC2 1-B3 antigen expression and scored as either positive (>10% of tumor cells) or negative.

Positive staining with the HPC2 1-B3 antibody strongly correlated with pancreatic or ampullary adenocarcinoma, with staining found in association with metastasis to liver, to lymph node and to colon. One of ten cholangiocarcinomas stained for HPC2 1-B3 and none of the gallbladder cancers stained for 1-B3.

The data indicates that the novel monoclonal antibody, HPC2 1-B3 can be used to distinguish primary cholangiocarcinoma, gallbladder cancer, and metastatic colon cancer, from metastatic pancreatic or ampullary carcinoma in liver. Separating these tumors has important implications for both patient prognosis and treatment. Further, detection of pancreatic cancer metastasis was not restricted to liver, and the antibody also detected this cancer in lymph nodes and colon, thus it is anticipated to allow detection of pancreatic adenocarcinoma metastasis throughout the body. The results are summarized below:

| | | Ampullary Cancer | | | | |
|---|---|---|---|---|---|---|
| | Pancreatic Cancer | (primary and | Cholangio- | Gallbladder | Colon Cancer | |
| | (metastasis) | metastasis) | carcionoma | Cancer | (metestasis) | Totals |
| Positive (%) | 11 (65) | 7 (70) | 1 (10) | 0 (0) | 1 (10) | 20 |
| Negative (%) | 6 (35) | 3 (30) | 9 (90) | 3 (100) | 9 (90) | 30 |
| Totals | 17 | 10 | 10 | 3 | 10 | 50 |

Without being bound by theory, the expression of the HPC2 1-B3 antigen in ampullary adenocarcinoma could be associated with the developmental and/or antatomical link between the ampula and the pancreas. The pancreatic duct and common bile duct merge and exit by way of the ampula.

### Example 9

### HPC2 1-B3 Monoclonal Antibodies for Detecting a Pancreatic Malignancy in a Subject or Confirming the Diagnosis of a Pancreatic Malignancy in a Subject or Monitoring Treatment Outcomes in a Subject with a Pancreatic Malignancy

This example describes additional assays for the use of HPC2 1-B3 monoclonal antibodies for the detection of a pancreatic adenocarcinoma in a subject. This example further describes the use of these antibodies to confirm the diagnosis of a pancreatic adenocarcinoma in a subject or to monitor treatment outcomes in a subject previously diagnosed with disease.

The antigen bound by the monoclonal antibody HPC2 1-B3 is expressed on pancreatic adenocarcinoma cells but not on pancreatic cells from healthy subjects. Thus, detection and quantitation of HPC2 1-B3 binding to cells in patients diagnosed with, or suspected of having IPMN or a pancreatic adenocarcinoma, can be used to detect a IPMN or pancreatic adenocarcinoma or confirm the diagnosis of IPMN or a pancreatic adenocarcinoma in a subject. A sample, such as a pancreatic biopsy specimen, serum, plasma, duct fluid, or a pancreatic tissue aspirate, is obtained from the patient diagnosed with, or suspected of having a pancreatic adenocarcinoma.

In this example a capture ELISAs is for detection of the HPC2 1-B3 antigens. After the genes that encode the HPC2 1-B3 antigen is identified, for example as described in Examples 3 and 4, the genetic sequence will be utilized to make recombinant protein. Based on the coding sequences of the gene, at least three distinct expression constructs will be used to generate amino- or carboxy-terminal 6× HIS tagged recombinant protein. The design of the expression constructs will take into account; the length of the open reading frame, putative hydrophobic and hydrophilic protein domains, recognized functional motives and post-translational modification signals. Qiagen® prokaryotic recombinant protein expression system is used in combination with E. coli BL-21 to render fast and robust expression levels from the expression constructs. However, if the size of the coding region exceeds 3 kb or contains putative mammalian post-translational modification signals, a eukaryotic expression system from Invitrogen® with the pMT/BiP/V5-His expression vector is used. D. melanogaster S2 cells are stably transfected with expression constructs to produce recombinant protein. S2 insect cells are particularly suited to produce high quantities of recombinant protein. Moreover, protein targeting (for example secretion) and post-translational modifications (for example glycosylation) are frequently conserved between invertebrate insect cells and mammalian cells. The HPC2 1-B3 antibody is used to monitor cross-reactivity with the recombinant protein. The isolation of our expressed proteins utilizes the HIS tag of the recombinant protein in combination with Ni-NTA columns.

A sandwich ELISA is performed to detect the HPC2 1-B3 antigen in the patient samples. A monoclonal HPC2 1-B3 antigen, such as the antibody HPC2 1-B3, is immobilized on the surface of a 96 well flat-bottomed plate by coating the plate with the antibody and incubating for 2 hours at room temperature. After washing the plate twice with 0.02% Tween PBS (T-PBS), the plate was blocked with 1% bovine serum albumin (BSA)-PBS to preclude nonspecific binding, then washed twice with T-PBS. The patient and control samples are added to the wells and incubated for approximately 15-20 hours. After washing with T-PBS three times, a second HPC2 1-B3 antigen antibody directly labeled with horseradish peroxidase (HRP) is added to the plate. After three more washes with T-PBS, 100 μl of 10,000-fold diluted Avidine-HRP solution (Biosource) is added and incubated 1 hour at room temperature. After three more washes with T-PBS, 100 μl of TMB solution (Pierce) and 100 μl of H₂O₂ are added and incubated for 5 minutes, followed by the addition of 100 μl of 2N H₂SO₄ to stop the color development. The levels of HPC2 1-B3 antigen are determined by measuring the OD value at 450 nm.

To facilitate quantitation of antigen levels in body fluids an antigen standard is used. This will be an antigen negative fluid and that same fluid spiked with various concentrations of antigen. The fluid for the standard will be from a patient with pancreatitis, and engineered to not contain any of the target antigens. Target antigens, if present in the pancreatitis fluid (identified by Western blot, dot blot, or during establishment of these assays) will be removed by passage of the fluid over immunoaffinity columns loaded with the HCP2 1-B3 antibody. Antigen for spiking of antigen negative duct fluid will be derived as indicated above from a recombinant source or a cell-derived native source.

### Example 10

### Additional Validation of the HPC2 1-B3 Antigen Capture ELISA Using Pancreatic Aspirates and Duct Fluid Specimens from Patients with Pancreatic Adenocarcinoma and Pancreatitis

The capture ELISA in Example 6 is further validated using duct fluid standards containing varied concentrations of antigen (positive specimen) and or no antigen (antigen-depleted negative control), and duct fluid specimens from patients with pancreatic cancer (confirmed antigen positive by immunohistochemistry) or with pancreatitis (confirmed antigen negative, or exhibiting staining of duct structures only, by immunohistochemistry). Specimens derived from patients with each type of pathology are included as part of the validation.

As part of this validation pancreatic fine needle aspirate specimens from patients with histologically confirmed adenocarcinoma or pancreatitis are also evaluated. Specimens from patients with each type of pathology will be included as part of this validation effort. As with duct fluid, fluid recovered from a fine needle aspirate from a patient with pancreatitis may need to be depleted of antigen to be used as a negative control specimen and spiked with antigen to serve as a standard.

### Example 11

### Additional Evaluation of Pancreatic Tissue Aspirates and Body Fluids for the Presence of HPC2 1-B3 Antigen

### Western Blot Analyses and Indirect ELISA:

Western blots are used to evaluate plasma, serum, duct fluid and pancreatic tissue aspirates from patients with pancreatitis and pancreatic adenocarcinoma for the presence of the HPC2 1-B3 antigens using the HPC2 1-B3 antibody as described in Example 2. Results will be correlated with those obtained by immunohistochemical assessment of tissue sections from these same patients. Indirect ELISAs are developed and used to evaluate duct fluid and pancreatic tissue aspirates from patients with pancreatitis and pancreatic adenocarcinoma for the presence of the HPC2 1-B3 antigen. Results will be correlated with those obtained by immunohistochemical assessment of tissue sections from these same patients.

Both the Western blot and indirect ELISAs will be initially conducted using duct fluid samples from patients with pancreatitis (negative controls) and samples from patients with pancreatic adenocarcinoma (positive controls). These will be the same specimens as used for capture ELISA validation, and as previously indicated, immunohistochemical methods will be employed to confirm antigen expression profiles in these tissues.

### Example 12

### HPC2 1-B3 Monoclonal Antibody for the Treatment of Pancreatic Adenocarcinoma

This example describes the use of HPC2 1-B3 monoclonal antibodies for the treatment of pancreatic adenocarcinoma that express HPC2 1-B3 antigen. In this example, patients diagnosed with a pancreatic adenocarcinoma are administered an immunoconjugate comprising a HPC2 1-B3 monoclonal antibody linked to a toxin, such as Pseudomonas exotoxin (PE). Preparation of PE immunoconjugates has been described (see, for example, U.S. Pat. No. 7,081,518 and U.S. Pre-Grant Publication Nos. 2005/0214304, 2005/0118182 and 2007/0189962). The HPC2 1-B3 immunoconjugate is administered by intravenous infusion in four doses, one dose per week. The dose of immunoconjugate administered to a patient varies depending on the weight and gender of the patient, and mode and time course of administration. Following treatment, patients are evaluated for cancer progression and other clinical signs of illness.

It will be apparent that the precise details of the methods or compositions described may be varied or modified without departing from the spirit of the described invention. We claim all such modifications and variations that fall within the scope and spirit of the claims below.

1. A hybridoma cell line HPC2 1-B3, as deposited with the American Type Culture Collection as ATCC accession number PTA-9400 (HPC2 1-B3 hybridoma) that produces a monoclonal antibody that specifically binds pancreatic neoplasia cells.

2. (canceled)

3. An isolated monoclonal antibody produced by the hybridoma cell line of claim 1, a chimeric form thereof, humanized form thereof, or an antigen binding fragment thereof, wherein the isolated monoclonal antibody, chimeric form thereof, humanized form thereof, or antigen binding fragment thereof specifically binds pancreatic neoplasia cells.

4. The isolated monoclonal antibody, chimeric form thereof, humanized form thereof, or antigen binding fragment thereof of claim 3, wherein the monoclonal antibody binds adenocarcinoma cells, IPMN cells or both.

5. (canceled)

6. The antigen binding fragment of claim 3, wherein the fragment is a scFV fragment, an scFV₂ fragment, an Fv fragment, an Fab fragment, or an F(ab′)₂ fragment.

7. The isolated monoclonal antibody, chimeric form thereof, humanized form thereof, or antigen binding fragment thereof of claim 3, conjugated to an effector molecule.

8. The isolated monoclonal antibody, chimeric form thereof, humanized form thereof, or antigen binding fragment thereof of claim 7, wherein the effector molecule is a toxin.

9. The isolated monoclonal antibody, chimeric form thereof, humanized form thereof, or antigen binding fragment thereof of claim 8, wherein the toxin comprises ricin A, abrin, diphtheria toxin or a subunit thereof, Pseudomonas exotoxin or a portion thereof, saporin, restrictocin or gelonin.

10. The isolated monoclonal antibody, chimeric form thereof, humanized form thereof, or antigen binding fragment thereof of claim 9, wherein the toxin comprises PE38, PE40, PE38KDEL, or PE38REDL.

11. The isolated monoclonal antibody, chimeric form thereof, humanized form thereof, or antigen binding fragment thereof of claim 3, labeled with a detectable agent.

12. The isolated monoclonal antibody, chimeric form thereof, humanized form thereof, or antigen binding fragment thereof of claim 11, wherein the detectable agent comprises an electron-dense compound, an enzyme, a fluorochrome, a hapten, or a radioisotope.

13. An isolated nucleic acid encoding the monoclonal antibody, chimeric form thereof, humanized form thereof, or antigen binding fragment thereof of claim 3.

14. The isolated nucleic acid of claim 13, operably linked to a promoter.

15. An isolated expression vector comprising the nucleic acid of claim 14.

16. An isolated host cell transformed with the expression vector of claim 15.

17. A composition comprising an effective amount of the monoclonal antibody, chimeric form thereof, humanized form thereof, or antigen binding fragment thereof of claim 3 and a carrier.

18. A method for detecting a pancreatic neoplasm cell comprising:
contacting a biological sample with the monoclonal antibody, chimeric form thereof, humanized form thereof, or antigen binding fragment thereof of claim 3 under conditions wherein an immune complex will form; and
detecting the formation of the immune complex,
wherein the presence of the immune complex detects the presence of the pancreatic neoplasm cell.

19. The method of claim 18, wherein the biological sample is a biopsy or a histological section from a subject.

20. The method of claim 18, wherein the neoplasm cell is an intraductal papillary mucinous neoplasm (IPMN) cell or a pancreatic adenocarcionoma cell.

21. The method of claim 20, wherein the pancreatic adenocarcinoma cell is a pancreatic ductal adenocarcinoma cell.

22. The method of claim 18, wherein the biopsy or histological section are from the liver of a subject.

23. The method of claim 18, wherein the biological sample is plasma sample, a serum sample, a duct fluid sample, or a tissue aspirate.

24. The method of claim 20, wherein the neoplasm cell is an IPMN cell, and wherein the method detects an IPMN cell that will develop into a pancreatic adenocarcinoma.

25. The method of claim 18, wherein detecting the presence of the pancreatic neoplasm cell indicates that the subject has metastatic pancreatic cancer.

26. A method for inhibiting the growth of a pancreatic neoplasm cell, comprising
contacting the cell with the monoclonal antibody, chimeric form thereof, humanized form thereof, or antigen binding fragment thereof of claim 3,
thereby inhibiting the growth of the pancreatic neoplasm cell.

27. The method of claim 26, wherein the pancreatic neoplasm cell is in vivo.

28. The method of claim 26, wherein the pancreatic neoplasm cell is an intraductal papillary mucinous neoplasm (IPMN) cell or a pancreatic adenocarcinoma cell.

29. The method of claim 26, wherein the pancreatic adenocarcinoma is a pancreatic ductal adenocarcinoma cell.

30. A method for treating a pancreatic neoplasm in a subject, comprising
administering to the subject a therapeutically effective amount of the monoclonal antibody, chimeric form thereof, humanized form thereof, or antigen binding fragment thereof of claim 3;
thereby treating the neoplasm in the pancreas of the subject.

31. The method of claim 30, wherein the pancreatic neoplasm is an intraductal papillary mucinous neoplasm (IPMN) or a pancreatic adenocarcinoma.

32. The method of claim 31, wherein the pancreatic adenocarcinoma is a pancreatic ductal adenocarcinoma.

33. The method of claim 31, wherein the pancreatic neoplasm has metastasized to the liver.

34. A method of isolating a pancreatic neoplasm cell, comprising:
contacting a biological sample comprising pancreatic cells with a first monoclonal antibody, chimeric form thereof, humanized form thereof, or antigen binding fragment thereof, wherein the first monoclonal antibody, chimeric form thereof, humanized form thereof, or antigen binding fragment thereof of is the antibody, chimeric form thereof, humanized form thereof or antigen binding fragment thereof of claim 3;
and isolating cells bound by the first monoclonal antibody, chimeric form thereof, humanized form thereof, or antigen binding fragment thereof;
thereby isolating the pancreatic neoplasm cell.

35. The method of claim 34, wherein the neoplasm cell is an intraductal papillary mucinous neoplasm (IPMN) cell or a pancreatic adenocarcinoma cell.

36. The method of claim 35, wherein the pancreatic adenocarcinoma is a pancreatic ductal adenocarcinoma cell.

37. The method of claim 34, further comprising
contacting the biological sample with a second antibody that specifically binds the first monoclonal antibody.

38. The method of claim 37, wherein the second antibody is coupled to a detectable agent.

39. The method of claim 38, wherein the detectable agent comprises an electron-dense compound, an enzyme, a fluorochrome, a hapten, or a radioisotope.

40. A kit for the detection or treatment of a pancreatic neoplasm cell or a pancreatic neoplasm cell antigen, comprising
a container comprising the monoclonal antibody, chimeric form thereof, humanized form thereof, or antigen binding fragment thereof of claim 3; and
instructions for using the kit.
